(19) United States
(12) Patent Application Publication
(10) Pub. No.: US 2026/0286350 A1
(43) Pub. Date: Sep. 24, 2026

(54) RNA-TARGETING SYSTEM AND USES THEREOF

(71) Applicant: The Regents of the University of California, Oakland, CA (US)

(72) Inventors: Daniel J. Brogan, La Jolla, CA (US); Tianqi Wang, La Jolla, CA (US); Fangying Chen, La Jolla, CA (US); Omar S. Akbari, La Jolla, CA (US)

(21) Appl. No.: 19/086,876

(22) Filed: Mar. 21, 2025

(51) Int. Cl. C12N 15/11; C12N 9/22; C12N 15/86

(52) U.S. Cl. CPC C12N 15/111; C12N 9/22; C12N 15/86; C07K 2319/09; C12N 2310/20; C12N 2750/14143

(57) ABSTRACT

Provided herein are compositions and methods of reducing the expression of a target sequence. Additionally provided are methods of regulating a target gene expression in a scalable manner. The compositions and methods include a recombinant CRISPR-Cas system using crRNA processing activity to reduce specific gene expression at protein and/or mRNA levels.

### FEDERALLY SPONSORED RESEARCH OR DEVELOPMENT

This invention was made with Government support under AI149161A and AI152071 awarded by the National Institutes of Health. The Government has certain rights in the invention.

### SEQUENCE LISTING

This application contains a Sequence Listing that has been submitted electronically as an XML file named “15670-0448001.XML.” The XML file, created on Mar. 14, 2025, is 40,526 bytes in size. The material in the XML file is hereby incorporated by reference in its entirety.

### TECHNICAL FIELD

This invention relates to the field of gene expression regulation. Specifically, the invention provides an RNA targeting system and methods for gene-specific repression of mRNA and protein expression.

### BACKGROUND

Gene expression, the process by which information from a gene is used to synthesize functional gene products (e.g., proteins or RNAs), is a complex and tightly regulated process.

It is controlled by a network of regulatory elements, including promoters, enhancers, silencers, and transcription factors. The interplay between these elements can be highly intricate, making it difficult to predict and manipulate gene expression accurately.

The genome-editing techniques and synthetic tools for regulating transcription factors are challenging to control and monitor, as their specificity relies on DNA-DNA or protein-DNA interactions in a crowded nucleus and some DNA regions are difficult to access.

Engineered enzymes, promoters, and terminators are also common synthetic gene expression regulation technologies, but these approaches can lack precision and are often complicated to engineer.

There is a need for innovative RNA-targeting technologies that support a wide range of applications, including RNA knockdown, translational regulation, transcript splicing, trafficking, and visualization. Additionally, there is a demand for RNA engineering technologies that are cost-effective, easy to design, highly specific and efficient, scalable, and capable of targeting multiple RNA molecules simultaneously.

### SUMMARY

This disclosure is related to recombinant CRISPR-Cas systems capable of reducing the expression of one or more target sequence, comprising: (a) a nucleic acid sequence encoding a CRISPR RNA (crRNA) comprising one or more direct repeats (DRs) and a polynucleotide sequence encoded by the one or more target sequence; and (b) a Cas peptide comprising a crRNA processing domain, wherein the recombinant CRISPR-Cas system does not reduce endogenous gene expression. In one aspect, the disclosure is related to recombinant CRISPR-Cas systems described herein that are capable of reducing a protein level by at least 40% or an RNA level by at least 40% compared to the protein level or RNA level without treatment the recombinant CRISPR-Cas system, wherein the protein or RNA are encoded by the target sequence. In some embodiments, the Cas peptide comprising a crRNA processing domain does not have enzymatic cleavage activity. In some embodiments, the one or more DR is or are positioned upstream, downstream, or both upstream and downstream of the polynucleotide sequence encoded by the target sequence. In some embodiments, the one or more DR is a single non-processed DR, a double non-processed DR (dDR), a double mature DR, or any combination thereof. In some embodiments, the single non-processed DR is about 25-45 nucleotides in length. In some embodiments, the single non-processed DR is about 35 nucleotides in length. In some embodiments, the one or more DR comprises a sequence at least 80%, 85%, 90%, 95%, 99%, or 100% identical to SEQ ID Nos.: 33, 34, or 35. In some embodiments, the crRNA optionally comprises a spacer sequence. In some embodiments, the spacer sequence is about 15-35 nucleotides in length. In some embodiments, the spacer sequence is about 22-24 nucleotides in length. In some embodiments, the target sequence comprises a reporter gene, GFP gene, mCherry gene, Cas 9 gene, CasRx gene, a toxin gene, a synthetic gene, or a transgene. In some embodiments, the Cas peptide further comprises a target coordination domain. In some embodiments, the target coordination domain is Cas 11, Cas 1, or any other domain of a target coordination activity. In some embodiments, the RNA encoded by the target sequence comprises messenger RNA (mRNA), synthetic RNA, single-stranded RNA, double-stranded RNA, non-coding RNA, small RNA, or microRNA. In some embodiments, the RNA encoded by the target sequence is messenger RNA (mRNA). In some embodiments, the Cas peptide comprise a variant of Cas7-11, Cas 12, Cas 13, Cas 5, Cas 6, or fragment thereof, wherein the variant does not have enzymatic cleavage activity. In some embodiments, the Cas peptide comprises the crRNA processing domain comprising Cas7.1 domain of a Cas7-11. In some embodiments, the Cas7-11 is DiCas7-11, Cbf Cas7-11, CjcCas7-11, CmaCas7-11, CsbCas7-11, DpbaCas7-11, FmCas7-11, GwCas7-11, HreCas7-11, HsmCas7-11, HvmCas7-11, OmCas7-11, SmCas7-11, SstCas7-11, or SybCas7-11. In some embodiments, the Cas7.1 is DiCas7.1, Cbf Cas7.1, CjcCas7.1, CmaCas7.1, CsbCas7.1, DpbaCas7.1, FmCas7.1, GwCas7.1, HreCas7.1, HsmCas7.1, HvmCas7.1, OmCas7.1, SmCas7.1, SstCas7.1, or SybCas7.1. In some embodiments, the Cas peptide comprises the crRNA processing domain comprising a DiCas7.1 domain. In some embodiments, the Cas peptide further comprises a dCas7.2, and/or a dCas7.2, where in the dCas7.2 has a substitution of aspartic acid (D) by alanine (A) at position 429, wherein the dCas7.3 has a substitution of aspartic acid (D) by alanine (A) at position 654. In some embodiments, the crRNA processing domain processes the crRNA to become a mature crRNA. In some embodiments, the mature crRNA comprises the polynucleotide sequence encoded by the target sequence and at least a portion of the direct repeats, wherein the at least a portion of the direct repeats are positioned upstream, downstream, or both upstream and downstream of polynucleotide sequence encoded by the target sequence. In some embodiments, the Cas peptide comprises one or more inactivated enzymatic cleavage domains comprising one or more amino acid mutation. In some embodiments, the Cas peptide optionally comprises one or more nucleus localization sequence (NLS). In some embodiments, the mature DR described herein is 10-20 nucleotides in length. In some embodiments, the mature DR described herein is about 15 nucleotides in length.

In one aspect, the disclosure is related to nucleic acids comprising a polynucleotide encoding recombinant CRISPR-Cas system, or fragments thereof, described herein. In some embodiments, the nucleic acid is a DNA or RNA.

In one aspect, the disclosure is related to vectors comprising one or more of the nucleic acids described herein.

In one aspect, the disclosure is related to cells comprising the vectors described herein. In some embodiments, the cell is a mammalian cell.

In one aspect, the disclosure is related to pharmaceutical compositions comprising the recombinant CRISPR-Cas system described herein.

In one aspect, the disclosure is related to methods of making the recombinant CRISPR-Cas system described herein comprising: (a) synthesize a nucleic acid sequence encoding CRISPR RNA (crRNA) comprising a nucleotide sequence encoding the one or more target sequence and one or more direct repeats (DRs); and (b) synthesize a nucleic acid comprising a nucleotide sequence encoding a Cas peptide or synthesize a recombinant Cas peptide using chemical and/or enzymatic reactions, wherein the recombinant Cas peptide comprises a crRNA processing domain. In some embodiments, the Cas peptide described herein optionally comprises one or more additional domains, wherein the domains are linked by one or more linker sequences. In some embodiments, the recombinant CRISPR-Cas system described herein further comprising purifying the synthesized nucleic acid sequence encoding crRNA. In some embodiments, the recombinant CRISPR-Cas system described herein further comprising purifying the synthesized nucleic acid comprising a nucleotide sequence encoding a Cas peptide or purifying the synthesized Cas peptide.

This disclosure further relates to methods of regulating one or more target sequence expression in a subject, the method comprising: (a) identifying the one or more target sequence; and (b) delivering, to the subject, a recombinant CRISPR-Cas system comprising: a nucleic acid sequence encoding CRISPR-RNA (crRNA) comprising one or more direct repeats and a nucleotide sequence coding the one or more target sequence; and a type III-E effector comprising a crRNA processing domain, wherein the type III-E effector and the nucleic acid sequence encoding crRNA are delivered at a ratio of about 1:5 to about 50:1. In some embodiments, the type III-E effector and the nucleic acid sequence encoding crRNA are delivered at a ratio of about 20:1, 18:1, 16:1, 15:1, 13:1, 11:1, 10:1, 9:1, 8:1, 7:1, 6:1, 5:1, 4:1, 3:1 2:1, 1:1, or 1:2. In some embodiments, the target sequence expression is reduced at mRNA and/or protein level. In some embodiments, the recombinant CRISPR-Cas system reduces the protein level by at least 40% and/or an RNA level by at least 40% compared to the protein level or RNA level without treatment of the recombinant CRISPR-Cas system, wherein the protein and RNA are encoded by the target sequence. In some embodiments, the recombinant CRISPR-Cas system reduces the protein level by at least 70% and/or an RNA level by at least 80% compared to the protein level or RNA level without treatment of the recombinant CRISPR-Cas system, wherein the protein and RNA are encoded by the target sequence. In some embodiments, the target sequence is coding or non-coding sequence. In some embodiments, the subject is a mammal, a human, a rodent, a mouse, a rat, a cell, a fruit fly, a mosquito, a microbial cell, or a bacterial cell. In some embodiments, delivering the recombinant CRISPR-Cas system comprises delivering a composition comprising one or more nucleic acid comprising one or more polynucleotide encoding the recombinant CRISPR-Cas system or fragments thereof. In some embodiments, delivering the recombinant CRISPR-Cas system comprises delivering a composition comprising a first polynucleotide encoding the crRNA and a second polynucleotide encoding the type III-E effector. In some embodiments, delivering the recombinant CRISPR-Cas system comprises delivering a nucleic acid comprising a polynucleotide encoding the crRNA and an amino acid sequence comprising the type III-E effector. In some embodiments, the type III-E effector comprises a crRNA processing domain comprising Cas7.1 of a Cas7-11. In some embodiments, the one or more DR is or are positioned upstream, downstream, or both upstream and downstream of the nucleotide sequence coding the target sequence. In some embodiments, the one or more DR is a single non-processed DR, a double non-processed DR (dDR), a double mature DR, or any combination thereof. In some embodiments, the target sequence is an endogenous or exogenous sequence. In some embodiments, the target sequence comprises messenger RNA (mRNA), synthetic RNA, single-stranded RNA, double-stranded RNA, non-coding RNA, small RNA, or microRNA. In some embodiments, the target sequence expression is regulated by an endogenous machinery comprising an exonuclease enzyme and/or exosome.

This disclosure also relates to methods of regulating a toxin gene expression in an insect, the method comprising delivering, to the insect, a recombinant CRISPR-Cas system comprising: a nucleic acid sequence encoding CRISPR-RNA (crRNA) comprising one or more direct repeats and a nucleotide sequence coding the toxin gene, wherein the toxin gene is lethal or nonlethal; and a type III-E effector comprising a crRNA processing domain, wherein the type III-E effector and the nucleic acid sequence encoding crRNA are delivered at a ratio of about 1:5 to about 50:1. In some embodiments, the toxin gene comprises bacterial toxins, diphtheria toxin, botulinum toxin, ricin, cholera toxin, pertussis toxin, Bacillus thuringiensis toxin, or aflatoxins. In some embodiments, the insect comprises a fruit fly, a mosquito, bedbug, a housefly, a clothes moth, a Japanese beetle, an aphid, a flea, a horsefly, a hornet, a butterfly, or a moth. In some embodiments, the toxin gene is dominant lethal. In some embodiments, the type III-E effector comprises a crRNA processing domain comprising Cas7.1 of a Cas7-11. In some embodiments, the one or more DR is or are positioned upstream, downstream, or both upstream and downstream of the nucleotide sequence coding the toxin gene.

This disclosure is related to methods of regulating expression of a gene engineered on an adeno-associated virus (AAV) vector, the method comprising delivering, to a subject, a recombinant CRISPR-Cas system comprising: a nucleic acid sequence encoding CRISPR-RNA (crRNA) comprising one or more direct repeats and a nucleotide sequence coding the gene; and a type III-E effector comprising a crRNA processing domain, wherein the type III-E effector and the nucleic acid sequence encoding crRNA are delivered at a ratio of about 1:5 to about 50:1. In some embodiments, the AAV vector comprise AAV1, AAV2, AAV4, AAV5, AAV6, AV6.2, AAV7, AAV8, AAV9, rh.10, rh.39, rh.43, or CSp3. In some embodiments, the subject is a mammal, a human, a rodent, a mouse, a rat, a cell, or a microbial cell. In some embodiments, the type III-E effector comprises a crRNA processing domain comprising Cas7.1 of a Cas7-11. In some embodiments, the one or more DR is or are positioned upstream, downstream, or both upstream and downstream of the nucleotide sequence coding the gene.

This disclosure further relates to methods of regulating expression of a gene in a culture container, the method comprising delivering, to a cell, a recombinant CRISPR-Cas system comprising: a nucleic acid sequence encoding CRISPR-RNA (crRNA) comprising one or more direct repeats and a nucleotide sequence coding the gene; and a type III-E effector comprising a crRNA processing domain, wherein the type III-E effector and the nucleic acid sequence encoding crRNA are delivered at a ratio of about 1:5 to about 50:1. In some embodiments, the cell comprises a mammalian cell, a microbial cell, a cell line, a primary cell, or a transgenic cell. In some embodiments, the type III-E effector comprises a crRNA processing domain comprising Cas7.1 of a Cas7-11. In some embodiments, the one or more DR is or are positioned upstream, downstream, or both upstream and downstream of the nucleotide sequence coding the gene.

### DESCRIPTION OF DRAWINGS

This application file contains at least one drawing executed in color. Copies of this patent or patent application publication with color drawing(s) will be provided by the Office upon request and payment of the necessary fee.

FIGS. 1A-1E shows CTRL construction, experimental design, and results of initial screening on variant CTRL constructs. (FIG. 1A) Schematic representation of (1) CTRL-DR design and processing. The dashed line ovals show the direct repeats (DRs) insertion sites. The triangle indicates the processing site on the DRs. (2) Target mRNA and processing. The coupling of 5′→3′ and 3′→5′ mRNA decay by exonucleases and exosomes following the CTRL-induced decapping and deadenylation results in the target-mRNA degradation. (FIG. 1B) qPCR analysis of CTRL-DR-EGFP/mCherry RNA knockdown with dDiCas7-11 (F (6, 14)=38.64, P<0.0001) and dDiCas7-11-NLS (F (6, 14)=88.00, P<0.0001) at a CTRL-effector:reporter molar ratio of 15:1. Error bars represent SEM (n=3). (FIG. 1C) Flow cytometry analysis of CTRL-DR-EGFP/mCherry protein knockdown with full-length dDiCas7-11 (F (6, 7)=342.7, P<0.0001) and dDiCas7-11-NLS (F (6, 7)=100.1, P<0.0001) at a CTRL-effector:reporter molar ratio of 15:1. Error bars represent SEM (n=3). (FIG. 1D) qPCR analysis of the RNA expression of CTRL-EGFP constructs engineered with unprocessed DRs (CTRL-dDR) or processed (or mature) DRs (CTRL-dmDR) and full-length dDiCas7-11 (F (9, 19)=24.71, P<0.0001) and dDiCas7-11-NLS (F (9, 20)=75.81, P<0.0001) at a 15:1 CTRL-effector:reporter molar ratio. Error bars represent SEM (n=3). (FIG. 1E) Flow cytometry protein expression analysis of CTRL-dDR-EGFP and CTRL-dmDR-EGFP constructs and full-length dDiCas7-11 (F (9, 30)=21.28, P<0.0001) at a 10:1 CTRL-effector:reporter molar ratio. Error bars represent SEM (n=4). For each analysis, significant differences in protein or RNA expression were determined by a one-way ANOVA and a Dunnett's multiple comparisons test between the CTRL-effectors and the DiCas7-11ΔCas7.1 control. P value style=GP (NS: P>=0.05; *: P<0.05; **: P<0.01; ***: P<0.001; ****: P<0.0001).

FIGS. 2A-2D show titration assays of truncated CTRL-effectors and mechanistic evidence in CTRL. (FIG. 2A) qPCR analysis of RNA expression in the truncated and full-length CTRL-effectors at different CTRL-effector:reporter molar ratios (10:1, 5:1, 2:1, 1:1). (FIG. 2B) Flow cytometry analysis of protein expression in the truncated and full-length CTRL-effectors at different CTRL-effector:reporter molar ratios (10:1, 5:1, 2:1, 1:1). (FIGS. 2C and 2D) Flow cytometry analysis of protein expression from mutated (FIG. 2C) Cas7.1 or (FIG. 2D) Cas7.1-7.2 with mutations in key residues (H43, Y55, N152) essential processing activities. Significant differences in RNA expression were determined using one-way ANOVA, Dunnett's multiple comparisons test, and Tukey's multiple comparisons test. Error bars represent SEM (n=4-6). P value style=GP (NS: P>=0.05; *: P<0.05; **: P<0.01; ***: P<0.001; ****: P<0.0001).

FIGS. 3A-3D show that CTRL regulated SpyCas9-NG and CasRx endonuclease activities. (FIG. 3A) Schematic of the CTRL knockdown of SpyCas9-NG and CasRx cleavage activity. The arrow indicates the processing site in the mDR sequence inserted downstream of the coding domain of SpyCas9-NG and CasRx. (FIG. 3B) Next-generation amplicon sequencing assessing SpyCas9-NG cleavage activity with CTRL effectors at a 15:1 CTRL-effector:CTRL-Cas9 molar ratio. The percentage of indels (insertions, deletions, and substitutions) within 8 bp spanning the predicted cleavage site was generated by MiSeq high-throughput DNA sequencer (Illumina) and analyzed with CRISPResso2. Error bars represent SEM (n=3). (FIG. 3C) qPCR assessing SpyCas9-NG mRNA expression by CTRL at a 15:1 CTRL-effector:CTRL-Cas9 molar ratio. Error bars represent SEM (n=3). (FIG. 3D) Assessing CasRx cleavage activity (targeting EGFP) by CTRL via qPCR analysis of EGFP mRNA expression. The CTRL-effector:CTRL-CasRx molar ratio is 15:1. Error bars represent SEM (n=3). Significant differences between all groups were calculated by one-way ANOVA and Dunnett's multiple comparisons test.

FIG. 4 shows the schematic representation of all CTRL-effectors and CTRL-reporters constructs. The triangles indicate the point mutations made in each construct compared to the wild type (DiCas7-11). The Addgene IDs are indicated after the constructs' description (all of the plasmids have been uploaded to and confirmed by Addgene).

FIG. 5 shows the schematic workflow of the assay design. CTRL-reporter and CTRL-effector plasmids were co-transfected into HEK293T cells and their expression levels were assessed with RT-qPCR and flow cytometry.

FIGS. 6A-6D show the processing activities of CTRL-5′DR(s) constructs. (FIG. 6A) A schematic representation of the primer amplification regions analyzed by three pairs of primers using qPCR analysis. F1-R1 spans the inserted DR(s) sequence. F2-R2 amplifies a EGFP coding domain closer to 5′ end. F3-R3 amplifies a EGFP coding domain closer to the 3′ end. (FIG. 6B) CTRL-5′DR(s)-EGFP expression with primers F1-R1. (FIG. 6C) CTRL-5′DR(s)-EGFP expression with primers F2-R2. (FIG. 6D) CTRL-5′DR(s)-EGFP expression with primers F3-R3.

FIG. 7 shows the truncated CTRL-effectors repressed mRNA expression of variant CTRL-EGFP reporters. The CTRL-effector:reporter molar ratio is 15:1. Significant differences in RNA expression were determined by a one-way ANOVA and a Dunnett's multiple comparisons test between the CTRL-effectors and the DiCas7-11ΔCas7.1 control. Error bars represent SEM (n=2).

FIGS. 8A-8B show the EGFP fluorescence expression. (FIG. 8A) In the titration assay, three replicates were conducted for each condition, demonstrating consistent fluorescence expression. The first replicate of each condition was displayed in the chart. (FIG. 8B) In the CTRL-CasRx assay, R1-3 represents replicates 1-3. The CTRL-effectors repressed CasRx expression and recovered EGFP expression.

FIGS. 9A-9D show the representative gating method for flow cytometry analysis of EGFP knockdown. (FIG. 9A) FSC vs SSC dot plots show the representative cell gating. (FIGS. 9B-9C) Further refinement of selected cells excludes clumped cells or debris, FSC width vs area (FIG. 9B) and SSC width vs area (FIG. 9C). (FIG. 9D) The selected EGFP-positive population.

FIG. 10 shows primers used herein for qPCR analysis.

FIG. 11 shows amino acid sequences and nucleotide sequences described in this disclosure.

### DETAILED DESCRIPTION

The disclosure describes genetic repressors (e.g., recombinant CRISPR-Cas systems or CTRL) that regulate synthetic or transgene expression. The provided genetic repressors are generalizable RNA-targeting systems utilizing the consistent crRNA array processing activity of type III-E Cas enzymes (e.g., Cas7-11 effectors or variants thereof), which does not require complex gRNA design and do not rely on gRNA binding to target sequences. This provides a platform for controlling the expression of specific genes without inducing mutations in the underlying DNA sequences. The recombinant CRISPR-Cas systems (e.g., CTRL) described herein are genetic tools for high-efficiency gene-specific knockdown, tunable regulation of gene expression, and gene-specific repression of mRNA and protein expression.

### RNA-Targeting CRISPR-Cas Systems

CRISPR-Cas systems are adaptive immune systems found in prokaryotes (bacteria and archaea) that protect against invading genetic elements like viruses and plasmids. CRISPR stands for Clustered Regularly Interspaced Short Palindromic Repeats, and Cas stands for CRISPR-associated proteins. These sequences are used to detect and destroy DNA from similar bacteriophages during subsequent infections. CRISPR is found in approximately 50% of sequenced bacterial genomes and nearly 90% of sequenced archaea. CRISPR-Cas systems fall into two classes. Class 1 systems use a complex of multiple Cas proteins to degrade foreign nucleic acids. Class 2 systems use a single large Cas protein for the same purpose. Class 1 is divided into types I, III, and IV; class 2 is divided into types II, V, and VI. The 6 system types are divided into 33 subtypes. Each type and most subtypes are characterized by a “signature gene” found almost exclusively in the category. Classification is also based on the complement of Cas genes that are present. CRISPR-Cas systems (e.g., CRISPR-Cas9) have been employed to edit genes by utilizing a guide RNA (gRNA) to direct the Cas protein (e.g., Cas9) to a specific DNA sequence.

The continuous evolution of CRISPR-Cas systems has led to the discovery and engineering of revolutionary genome engineering technologies. Despite its revolutionary potential, CRISPR-Cas technology faces several challenges, including off-target effects, delivery challenges of CRISPR components (e.g., gRNA and Cas9), regulatory difficulties, and uncertain effectiveness and safety. A detailed description of CRISPR-Cas systems can be found, e.g., in Wright A V, et al. “Biology and Applications of CRISPR Systems: Harnessing Nature's Toolbox for Genome Engineering”. Cell 164 (1-2): 29-44 (January 2016); Makarova K S, et al. “Evolutionary classification of CRISPR-Cas systems: a burst of class 2 and derived variants”. Nature Reviews Microbiology 18 (1): 67-83 (December 2019).

Unlike the more commonly known DNA-targeting CRISPR-Cas systems, which edit DNA, RNA-targeting systems focus on RNA molecules. RNA-targeting CRISPR-Cas systems are categorized as either type III (e.g., Cas7-11, Csm, or Cmr) or type VI (e.g., Cas13), where type III systems predominantly utilize multisubunit effector and type VI systems encode a single polypeptide. RNA-targeting CRISPR-Cas systems possess two different RNAse activities: target cleavage and CRISPR-RNA (crRNA) array processing. Target cleavage is accomplished through ternary complex formation, where the spacer hybridizes with its complement and utilizes divalent cations to coordinate catalysis. However, crRNA array processing operates independently of metal ions. crRNA array processing occurs when a CRISPR effector recognizes the distinct sequence and secondary structure of the crRNA direct repeats (DRs) and cuts the 5′ and/or 3′ end of the crRNA, resulting in a mature crRNA molecule. crRNA array processing is consistent regardless of the programmed spacer and, therefore differs from target cleavage, which is inherently variable across spacers. DRs, in bacterial species, are short, palindromic nucleotide sequences that are interspersed with unique spacer sequences derived from previous viral or plasmid invaders.

The discovery of RNA-targeting CRISPR-Cas systems has enabled the development of programmable RNA-targeting systems for effective gene knockdown. Gene knockdown enables the characterization of gene expression, signaling pathways, and molecular interactions. Robust RNA knockdown has been achieved with multiple type III and type VI systems. These systems can theoretically target any gene of interest, but the on-target efficacy is variable and requires large screens to optimize gRNA design. Cas13 also exhibits a nonspecific RNase activity useful for diagnostics but can have toxic off-target activity. These characteristics limit the precision of gene knockdown at the post-transcriptional level and present an opportunity to develop improved gene knockdown technologies.

### Type III-E CRISPR-Cas System

Type III-E CRISPR-Cas system (e.g., Cas7-11) has changed the understanding of Cas protein evolution. Type III-E effectors (e.g., Cas7-11 effectors) are categorized as class 1 CRISPR-Cas system, which typically utilize a multiprotein complex to accomplish DNA-and/or RNA-targeting. Type III-E Cas effectors accomplish programmable RNA-targeting by employing a single, large protein composed of four Cas7-like domains (Cas7.1 (crRNA processing domain), Cas7.2 (enzymatic cleavage domain), Cas7.3 (enzymatic cleavage domain), and Cas7.4), one Cas11 domain (target coordination domain), and a large insertion (INS) domain, hence being named Cas7-11 (or gRAMP for giant Repeat Associated Mysterious Protein). Cas7-11 has been identified in various bacterial species, and examples of bacterial species include, but are not limited to, Desulfonema ishimotonii (DiCas7-11), Candidatus Brocadia fulgida (CbfCas7-11), Candidatus Jettenia caeni (CjcCas7-11), Candidatus Methanoperedens arcticus (CmaCas7-11), Candidatus Scalindua brodae (CsbCas7-11), Desulfobulbus propionicus (DpbaCas7-11), Ferroglobus placidus (FmCas7-11), Geobacter sulfurreducens (GwCas7-11), Halorubrum lacusprofundi (HreCas7-11), Halobacterium salinarum (HsmCas7-11), Halorubrum vacuolatum (HvmCas7-11), Opitutaceae bacterium (OmCas7-11), Sulfolobus metallicus (SmCas7-11), Sulfolobus solfataricus (SstCas7-11), and Synechococcus sp (SybCas7-11).

TABLE 1
Amino acid sequences for wildtype DiCas7-11 effectors and the nucleotide
sequence for the corresponding direct repeats (DRs)

| | SEQ | |
|---|---|---|
| Domain | ID No. | Sequences |
| DiCas7-11 | 27 | MTTTMKISIEFLEPFRMTKWQESTRRNKNNKEFVRG |
| (NCBI | | QAFARWHRNKKDNTKGRPYITGTLLRSAVIRSAENL |
| Accession: | | LTLSDGKISEKTCCPGKFDTEDKDRLLQLRQRSTLRW |
| 8D1V_A) | | TDKNPCPDNAETYCPFCELLGRSGNDGKKAEKKDW |
| 1601 aa | | RFRIHFGNLSLPGKPDFDGPKAIGSQRVLNRVDFKSG |
| | | KAHDFFKAYEVDHTRFPRFEGEITIDNKVSAEARKLL |
| | | CDSLKFTDRLCGALCVIRFDEYTPAADSGKQTENVQ |
| | | AEPNANLAEKTAEQIISILDDNKKTEYTRLLADAIRSL |
| | | RRSSKLVAGLPKDHDGKDDHYLWDIGKKKKDENSV |
| | | TIRQILTTSADTKELKNAGKWREFCEKLGEALYLKSK |
| | | DMSGGLKITRRILGDAEFHGKPDRLEKSRSVSIGSVLK |
| | | ETVVCGELVAKTPFFFGAIDEDAKQTDLQVLLTPDNK |
| | | YRLPRSAVRGILRRDLQTYFDSPCNAELGGRPCMCKT |
| | | CRIMRGITVMDARSEYNAPPEIRHRTRINPFTGTVAEG |
| | | ALFNMEVAPEGIVFPFQLRYRGSEDGLPDALKTVLK |
| | | WWAEGQAFMSGAASTGKGRFRMENAKYETLDLSDE |
| | | NQRNDYLKNWGWRDEKGLEELKKRLNSGLPEPGNY |
| | | RDPKWHEINVSIEMASPFINGDPIRAAVDKRGTDVVT |
| | | FVKYKAEGEEAKPVCAYKAESFRGVIRSAVARIHME |
| | | DGVPLTELTHSDCECLLCQIFGSEYEAGKIRFEDLVFE |
| | | SDPEPVTFDHVAIDRFTGGAADKKKFDDSPLPGSPAR |
| | | PLMLKGSFWIRRDVLEDEEYCKALGKALADVNNGL |
| | | YPLGGKSAIGYGQVKSLGIKGDDKRISRLMNPAFDET |
| | | DVAVPEKPKTDAEVRIEAEKVYYPHYFVEPHKKVER |
| | | EEKPCGHQKFHEGRLTGKIRCKLITKTPLIVPDTSNDD |
| | | FFRPADKEARKEKDEYHKSYAFFRLHKQIMIPGSELR |
| | | GMVSSVYETVTNSCFRIFDETKRLSWRMDADHQNVL |
| | | QDFLPGRVTADGKHIQKFSETARVPFYDKTQKHFDIL |
| | | DEQEIAGEKPVRMWVKRFIKRLSLVDPAKHPQKKQD |
| | | NKWKRRKEGIATFIEQKNGSYYFNVVTNNGCTSFHL |
| | | WHKPDNFDQEKLEGIQNGEKLDCWVRDSRYQKAFQ |
| | | EIPENDPDGWECKEGYLHVVGPSKVEFSDKKGDVIN |
| | | NFQGTLPSVPNDWKTIRTNDFKNRKRKNEPVFCCED |
| | | DKGNYYTMAKYCETFFFDLKENEEYEIPEKARIKYKE |
| | | LLRVYNNNPQAVPESVFQSRVARENVEKLKSGDLVY |
| | | FKHNEKYVEDIVPVRISRTVDDRMIGKRMSADLRPCH |
| | | GDWVEDGDLSALNAYPEKRLLLRHPKGLCPACRLFG |
| | | TGSYKGRVRFGFASLENDPEWLIPGKNPGDPFHGGPV |
| | | MLSLLERPRPTWSIPGSDNKFKVPGRKFYVHHHAWK |
| | | TIKDGNHPTTGKAIEQSPNNRTVEALAGGNSFSFEIAF |
| | | ENLKEWELGLLIHSLQLEKGLAHKLGMAKSMGFGSV |
| | | EIDVESVRLRKDWKQWRNGNSEIPNWLGKGFAKLKE |
| | | WFRDELDFIENLKKLLWFPEGDQAPRVCYPMLRKKD |
| | | DPNGNSGYEELKDGEFKKEDRQKKLTTPWTPWA |
| DiCas7.1 | 12 | MTTTMKISIEFLEPFRMTKWQESTRRNKNNKEFVRG |
| | | QAFARWHRNKKDNTKGRPYITGTLLRSAVIRSAENL |
| | | LTLSDGKISEKTCCPGKFDTEDKDRLLQLRQRSTLRW |
| | | TDKNPCPDNAETYCPFCELLGRSGNDGKKAEKKDW |
| | | RFRIHFGNLSLPGKPDFDGPKAIGSQRVLNRVDFKSG |
| | | KAHDFFKAYEVDHTRFPRFEGEITIDNKVSAEARKLL |
| | | CDSLKFTDRLCGALCVIRF |
| DiCas7.2 | 28 | KETVVCGELVAKTPFFFGAIDEDAKQTDLQVLLTPDN |
| | | KYRLPRSAVRGILRRDLQTYFDSPCNAELGGRPCMC |
| | | KTCRIMRGITVMDARSEYNAPPEIRHRTRINPFTGTVA |
| | | EGALFNMEVAPEGIVFPFQLRYRGSEDGLPDALKTVL |
| | | KWWAEGQAFMSGAASTGKGRFRMENAKYETLDL |
| DiCas7.3 | 29 | WHEINVSIEMASPFINGDPIRAAVDKRGTDVVTFVKY |
| | | KAEGEEAKPVCAYKAESFRGVIRSAVARIHMEDGVP |
| | | LTELTHSDCECLLCQIFGSEYEAGKIRFEDLVFESDPEP |
| | | VTFDHVAIDRFTGGAADKKKFDDSPLPGSPARPLML |
| | | KGSFWIRRDVLEDEEYCKALGKALADVNNGLYPLGG |
| | | KSAIGYGQVKSLGIKG |
| DiCas7.4 | 30 | LTGKIRCKLITKTPLIVPDTSNDDFFRPADKEARKEKD |
| (5′ | | EYHKSYAFFRLHKQIMIPGSELRGMVSSVYETVTNSC |
| terminal | | FRIFDETKRLSWRM |
| portion) | | |
| DiCas7.4 | 31 | RTVDDRMIGKRMSADLRPCHGDWVEDGDLSALNAY |
| (3′ | | PEKRLLLRHPKGLCPACRLFGTGSYKGRVRFGFASLE |
| terminal | | NDPEWLIPGKNPGDPFHGGPVMLSLLERPRPTWSIPG |
| portion) | | SDNKFKVPGRKFYVHHHAWKTIKDGNHPTTGKAIEQ |
| | | SPNNRTVEALAGGNSFSFEIAFENLKEWELGLLIHSLQ |
| | | LEKGLAHKLGMAKSMGFGSVEIDVESVRLRK |
| DiCas11 | 32 | NLAEKTAEQIISILDDNKKTEYTRLLADAIRSLRRSSK |
| | | LVAGLPKDHDGKDDHYLWDIGKKKKDENSVTIRQIL |
| | | TTSADTKELKNAGKWREFCEKLGEALYLKSKDMS |
| Direct | 33 | GGTTGgaaagccggttttctttgatGTCACGGAAC |
| repeats | | |
| (DRs for | | |
| DiCas7-11) | | |

Type III-E effectors are capable of programmable RNA knockdown in prokaryotic and eukaryotic cells, and do not exhibit RNA collateral cleavage activity, which consistently limits the applications of CRISPR-Cas13 systems. Using prokaryotic expression, Cas7-11 target binding activates a downstream caspase activity that results in cell dormancy or death, and appears to serve as the primary immune response for type III-E CRISPR-Cas systems. Therefore, when type III-E effectors are expressed independently of their protease signaling pathway, they operate as highly specific ribonucleases, and their unique architecture provides a pathway for further engineering.

Effector size is important when engineering CRISPR-Cas systems, and efforts have been made to generate compact effectors for almost all therapeutically relevant CRISPR-Cas systems. Usually, Class I CRISPR-Cas systems are extremely challenging to encode in therapeutic vectors since they require co-expression of multiple independent proteins, which highlights the advantage of using Cas7-11 instead. However, type III-E effectors are the largest single-effector Cas protein subtype discovered to date, which is a limitation for therapeutic applications, such as adeno-associated virus (AAV) delivery. Cas13 effectors, such as the compact Cas13d subtype, are more feasible for packaging but severely limited by collateral cleavage of ssRNA. Recently, a small Cas7-11 (Cas7-11S) protein was engineered through deletion of the INS domain and replacement with a GS-linker. However, the unique architecture of Cas7-11 characterized in the present disclosure demonstrates a unique modularity of type III-E effectors that can be exploited for engineering.

CRISPR-Cas Transgenic Repressible eLement (CTRL)

Provided herein are recombinant CRISPR-Cas systems (e.g., CTRL) capable of reducing the expression of a synthetic gene or transgene utilizing the direct repeats processing activity of CRISPR-Cas effectors (e.g., CRISPR-Cas7-11 effectors) to site-specifically target synthetic mRNA molecules. The recombinant CRISPR-Cas systems (e.g., CTRL) described herein encoded multiple permutations of crRNA direct repeats (DRs) into mRNA molecules. As described in the present disclosure, the recombinant CRISPR-Cas systems (e.g., CTRL) consist of (a) a CRISPR RNA (crRNA) comprising one or more direct repeats (DRs) and a polynucleotide sequence encoded by the synthetic gene or transgene; and (b) a Cas peptide comprising a crRNA processing domain, wherein the recombinant CRISPR-Cas system does not reduce endogenous gene expression.

The recombinant CRISPR-Cas systems (e.g., CTRL) described herein can reduce a protein level by at least 40% (e.g., about 40%-100%, about 50%-100%, about 60%-100%, about 70%-100%, about 80%-100%, about 90%-100%, about 40%, about 45%, about 50%, about 55%, about 60%, about 65%, about 70%, about 75%, about 80%, about 85%, about 90%, about 95%, about 99%, or about 100%) or an RNA level by at least 40% (e.g., about 40%-100%, about 50%-100%, about 60%-100%, about 70%-100%, about 80%-100%, about 90%-100%, about 40%, about 45%, about 50%, about 55%, about 60%, about 65%, about 70%, about 75%, about 80%, about 85%, about 90%, about 95%, about 99%, or about 100%) compared to the protein level or RNA level without treatment the recombinant CRISPR-Cas system, wherein the protein and RNA are encoded by the synthetic gene or transgene. In some embodiments, the recombinant CRISPR-Cas systems (e.g., CTRL) described herein can reduce the protein level by at least 40% (e.g., about 40%-100%, about 50%-100%, about 60%-100%, about 70%-100%, about 80%-1000%, about 900%-100%, about 40%, about 45%, about 50%, about 55%, about 60%, about 65%, about 70%, about 75%, about 80%, about 85%, about 90%, about 95%, about 99%, or about 100%) and the RNA level by at least 40% (e.g., about 40%-100%, about 50%-100%, about 60%-100%, about 70%-100%, about 80%-100%, about 90%-100%, about 40%, about 45%, about 50%, about 55%, about 60%, about 65%, about 70%, about 75%, about 80%, about 85%, about 90%, about 95%, about 99%, or about 100%), wherein the protein and RNA are encoded by the synthetic gene or transgene. In some embodiments, the recombinant CRISPR-Cas systems (e.g., CTRL) described herein can reduce the protein level by at least 70% (e.g., about 70%-100%, about 75%-100%, about 80%-100%, about 85%-100%, about 90%-100%, about 95%-100%, about 70%-80%, about 75%-95%, about 80%-90%, about 70%, about 75%, about 80%, about 85%, about 90%, about 95%, about 99%, or about 100%), while the RNA level was reduced by at least 80% (e.g., about 80%-100%, about 85%-100%, about 90%-100%, about 95%-100%, about 85%-95%, about 80%-90%, about 80%, about 85%, about 90%, about 95%, about 99%, or about 100%. In some embodiments, the recombinant CRISPR-Cas systems (e.g., CTRL) described herein can reduce the protein level by at least 70% (e.g., about 70%-100%, about 75%-100%, about 80%-100%, about 85%-100%, about 90%-100%, about 95%-100%, about 70%-80%, about 75%-95%, about 80%-90%, about 70%, about 75%, about 80%, about 85%, about 90%, about 95%, about 99%, or about 100%), while the RNA level was reduced less than 80% (e.g., about 0-79.9%, about 0-70%, about 0-60%, about 0-50%, about 0-40%, about 0-30%, about 0-20%, about 0-10%, about 0-5%, about 10%-70%, about 20%-60%, about 30%-50%, about 40%-70%, about 0, about 1%, about 5%, about 10%, about 15%, about 20%, about 25%, about 30%, about 35%, about 40%, about 45%, about 50%, about 55%, about 60%, about 65%, about 70%, or about 75%), wherein the protein and RNA are encoded by the synthetic gene or transgene.

CRISPR RNA (crRNA)

Provided herein are nucleic acid sequence (e.g., DNA or RNA) encoding crRNA that include a promoter region (e.g., CMV promoter or U6 promoter), one or more CTRL direct repeats (DRs), a target sequence (e.g., a reporter gene, a functional gene (e.g., Cas9 or CasRx), a synthetic gene, or a transgene), and a transcription terminator (e.g., growth hormone (bGH) terminator). In some embodiments, the nucleic acid sequence encoding crRNA is synthetic.

A promoter can be a DNA sequence recognized by enzymes/proteins in a mammalian cell required to initiate the transcription of a linked coding sequence. A promoter typically refers to e.g., a nucleotide sequence to which an RNA polymerase and/or any associated factor binds and at which transcription is initiated. The promoter can be constitutive, inducible, or tissue-specific. The promoter can be an exogenous promoter operably linked to an isolated nucleic acid. The promoter can also be a genomic sequence where the promoter is proximal to a transcription start site and at least partially controls expression of the associated gene product. A promoter within the genome can be either proximal (e.g., within 2000 nucleotides) or distal (e.g., greater than 2000 nucleotides) from a transcription start site. Non-limiting examples of promoters include a U6 promoter (e.g., human U6 or mouse U6 promoter), a CMV promoter, a T7 promoter, or an adeno-associated virus (AAV) promoter. In some embodiments, the promoter region includes a CMV promoter. In some embodiments, the promoter region includes an U6 promoter.

A transcription terminator can be a section of nucleic acid sequence that marks the end of a gene or operon in genomic DNA during transcription. This sequence mediates transcriptional termination by providing signals in the newly synthesized transcript RNA that trigger processes which release the transcript RNA from the transcriptional complex. In some embodiments, the transcription terminator is bGH polyA signal.

Direct repeats (DRs), identified in bacterial species, are short, palindromic nucleotide sequences that are interspersed with unique spacer sequences derived from previous viral or plasmid invaders. The DRs disclosed herein can be single non-processed DR (CTRL-DR), double non-processed DR (CTRL-dDR), double mature DR (CTRL-dmDR), or any combination thereof. In some embodiments, the one or more (e.g., 1, 2, 3, 4, or 5) DRs are positioned upstream, downstream, or both upstream and downstream of the nucleotide sequence coding the target sequence. In some embodiments, the one or more (e.g., 1, 2, 3, 4, or 5) DRs are a single non-processed DR, a double non-processed DR (dDR), a double mature DR, or any combination thereof. In some embodiments, mature DR is double mature DR.

A mature direct repeat is the direct repeat sequence found within the CRISPR RNA array after the precursor CRISPR RNA (pre-crRNA) has been processed into mature CRISPR RNA (crRNA). Mature crRNA described herein contains a polynucleotide sequence encoded by the synthetic gene or transgene and at least a portion of the direct repeats. In some embodiments, mature crRNA contains a portion of, or the entire direct repeats positioned upstream of polynucleotide sequence encoded by the synthetic gene or transgene. In some embodiments, mature crRNA contains a portion of, or the entire direct repeats positioned downstream of polynucleotide sequence encoded by the synthetic gene or transgene. In some embodiments, mature crRNA contains a portion of, or the entire direct repeats positioned both upstream and downstream of polynucleotide sequence encoded by the synthetic gene or transgene.

Double DRs (e.g., double non-processed DR (dDR) or double mature DR (dmDR)) described herein refer to two single DRs (e.g., two single non-processed DRs, two single processed DRs, two different single DRs, or two identical single DRs) in the same direction follow one another. In some embodiments, the non-processed DR (e.g., single or double non-processed DR) includes one or more stem-loop structure. In some embodiments, the two single DRs (e.g., identical or not identical, processed or non-processed) are separated by a spacer. In some embodiments, the mature DR (or double mature DR) described herein is linear. In some embodiments, the mature DR (or double mature DR) described herein includes two processed DR (e.g., identical or not identical separated by a spacer.

The single non-processed DR is about 15-45 nucleotides (e.g., about 23-35 nt, about 15, 18, 20, 21, 22, 23, 24, 25, 26, 27, 28, 29, 30, 31, 32, 33, 34, 35, 36, 37, 38, 39, 40, 41, 42, 43, 44, or 45 nt) in length. In some embodiments, the single non-processed DR is about 35 nucleotides in length. The mature DR (or double mature DR) is 10-20 nucleotides (e.g., about 10, 11, 12, 13, 14, 15, 16, 17, 18, 19, or 20 nt) in length. In some embodiments, the mature DR (e.g., double mature DR) is about 15 nucleotides in length. In some embodiments, the non-processed DR comprises a sequence at least 80%, 85%, 90%, 95%, 99%, or 100% identical to SEQ ID No.: 33 or 34. In some embodiments, the mature DR comprises a sequence at least 80%, 85%, 90%, 95%, 99%, or 100% identical to SEQ ID No.: 35.

TABLE 2
Nucleotide sequences for CTRL-DRs

| | SEQ | |
|---|---|---|
| | ID | |
| DR | No. | Sequence |
| single non- | 33 | GGTTGgaaagccggttttctttgatGTCAC |
| processed DR | | GGAAC |
| (CTRL-DR) | | |
| double non- | 34 | GGTTGgaaagccggttttctttgatGTCAC |
| processed DR | | GGAACCTACCTGGTAGCCCTTGTATTTGGT |
| (CTRL-dDR) | | TGgaaagccggttttctttgatGTCACGGA |
| double mature | 35 | ACttgatGTCACGGAACCTACCTGGTAGCC |
| DR (CTRL- | | CTTGTATTTttgatGTCACGGAAC |
| dmDR) | | |

As described herein, DR (or CTRL DR) can be positioned upstream, downstream, or both upstream and downstream of the polynucleotide sequence encoded by the synthetic gene or transgene. In some embodiments, one or more (e.g., 1, 2, 3, 4, or 5) DRs are positioned upstream of the polynucleotide sequence encoded by the synthetic gene or transgene. In some embodiments, one or more (e.g., 1, 2, 3, 4, or 5) DRs are positioned downstream of the polynucleotide sequence encoded by the synthetic gene or transgene. In some embodiments, one or more (e.g., 1, 2, 3, 4, or 5) DRs are positioned upstream and one or more (e.g., 1, 2, 3, 4, or 5) DRs are positioned downstream of the polynucleotide sequence encoded by the synthetic gene or transgene. In some embodiments, the one or more DRs upstream of the polynucleotide sequence encoded by the synthetic gene or transgene are positioned downstream of the 5′ cap and upstream of the Kozak consensus sequence. In some embodiments, the one or more DRs downstream of the polynucleotide sequence encoded by the synthetic gene or transgene are positioned downstream of the stop codon and upstream of the 3′ UTR and poly-A residues. The Kozak consensus sequence is a specific sequence of nucleotides found in eukaryotic mRNA that plays a crucial role in the initiation of translation.

This sequence helps ribosomes identify the correct start codon (AUG) to begin protein synthesis. The Kozak sequence is typically represented as: 5′-(gcc)gccRccAUGG-3′5′-(gcc)gccRccAUGG-3′ (SEQ ID No.: 23) (Kozak M. “An analysis of 5′-noncoding sequences from 699 vertebrate messenger RNAs”. Nucleic Acids Res 15 (20): 8125-8148 (October 1987)).

In some embodiments, one single non-processed DR is positioned upstream (CTRL-5′ DR) of the polynucleotide sequence encoded by the synthetic gene or transgene. In some embodiments, one single non-processed DR is positioned downstream (CTRL-3′ DR) of the polynucleotide sequence encoded by the synthetic gene or transgene. In some embodiments, one single non-processed DR is positioned upstream and one single non-processed DR is positioned downstream (CTRL-5′+3′ DR) of the polynucleotide sequence encoded by the synthetic gene or transgene. In some embodiments, double non-processed DR is positioned upstream (CTRL-5′ dDR) of the polynucleotide sequence encoded by the synthetic gene or transgene. In some embodiments, double non-processed DR is positioned downstream (CTRL-3′ dDR) of the polynucleotide sequence encoded by the synthetic gene or transgene. In some embodiments, double non-processed DR is positioned upstream and double non-processed DR is positioned downstream (CTRL-5′+3′ dDR) of the polynucleotide sequence encoded by the synthetic gene or transgene. In some embodiments, double mature DR (or mature DR) is positioned upstream (CTRL-5′ dmDR) of the polynucleotide sequence encoded by the synthetic gene or transgene. In some embodiments, double mature DR is positioned downstream (CTRL-3′ dmDR) of the polynucleotide sequence encoded by the synthetic gene or transgene. In some embodiments, double mature DR is positioned upstream and double mature DR is positioned downstream (CTRL-5′+3′ dmDR) of the polynucleotide sequence encoded by the synthetic gene or transgene.

### Target Sequence

The nucleic acid sequence encoding any crRNA disclosed herein can include a polynucleotide encoding a target sequence. The target sequence can encode coding sequences or non-coding sequences. In some embodiments, the target sequences encode proteins (e.g., fluorescence proteins, CRISPR-Cas proteins, transcription factors, or protein toxins). In some embodiments, the target sequence encodes RNAs (e.g., messenger RNA (mRNA), synthetic RNA, single-stranded RNA, double-stranded RNA, non-coding RNA, small RNA, or microRNA). The target sequence(s) described herein include, but are not limited to, a reporter gene, a functional gene (e.g., Cas9 or CasRx), a synthetic gene, or a transgene). The target sequence(s) described herein may be experimentally determined or found on one of many public databases, such as Addgene (www.addgene.org).

In some embodiments, the crRNA described herein includes one or more (e.g., 1, 2, 3, 4, 5, or more) target sequences. In some embodiments the target sequence can include a reporter gene. In some embodiments, the target sequence can encode one or more reporter genes (e.g., GFP gene or mCherry gene). Reporter genes are often used as an indication of whether a certain gene has been taken up by or expressed in the cell or organism population. Commonly used reporter genes that induce visually identifiable characteristics usually involve fluorescent and luminescent proteins. Examples of a reporter gene can include, but are not limited to, the genes encoding fluorescent proteins (e.g., GFP, dsRed, YFP, RFP, mCherry, and EGFP), luciferase (e.g., firefly luciferase, renilla luciferase), β-Galactosidase (e.g., LacZ,), HaloTag, and GUS (0-Glucuronidase). (Lippincott-Schwartz and Patterson, Sci. 300: 87-91; Contag and Bachmann, Annu. Rev. Biomed. Eng. 4: 235-260; Salehi et al, Hum. Gene Ther. 20 (1):21-30; Giepmans et al, Sci. 312 (5771): 217-224; Marathe and McEwen, Gene. 154 (1): 105-107). In some embodiments, a reporter gene can be EGFP or mCherry.

In some embodiments, the target sequence encodes a Cas 9 gene or CasRx gene that has downstream regulatory functions. In some embodiments, the target sequence is a toxin gene, e.g., bacterial toxins, diphtheria toxin, botulinum toxin, ricin, cholera toxin, pertussis toxin, Bacillus thuringiensis (Bt) toxin, or aflatoxins.

The crRNA described herein optionally includes a spacer sequence. In some embodiments, the crRNA described herein does not include a spacer sequence downstream of a single DR (e.g., CTRL-5′ DR, CTRL-3′ DR, CTRL-5′+3′ DR, CTRL-5′ mDR, CTRL-3′ mDR, or CTRL-5′+3′ mDR). In some embodiments, the spacer sequence is about 15-35 nucleotides (e.g., about 15, 16, 17, 18, 19, 20, 21, 22, 23, 24, 25, 26, 27, 28, 29, 30, 31, 32, 33, 34, or 35 nt) in length. In some embodiments, the spacer sequence is about 22-24 nucleotides (e.g., about 22, 23, or 24 nt) in length. In some embodiments, the spacer sequence is 22 nucleotides in length. In some embodiments, the spacer sequence is downstream of a CTRL-5′ DR or a CTRL-5′ mDR. In some embodiments, the spacer sequence is downstream of a CTRL-3′ DR or a CTRL-3′ mDR. In some embodiments, the spacer sequence is downstream of both DRs of a CTRL-5′+3′ DR or a CTRL-5′+3′ mDR. In some embodiments, the spacer sequence is between the two DRs of a CTRL-5′ dDR or a CTRL-5′ dmDR. In some embodiments, the spacer sequence is between the two DRs of a CTRL-3′ dDR or a CTRL-3′ dmDR. In some embodiments, the spacer sequence is between the two DRs of a CTRL-5′+3′ dDR or a CTRL-5′+3′ dmDR.

### Cas Peptide

The Cas peptides described herein include a crRNA processing domain and do not have enzymatic cleavage activity. In some embodiments, the Cas peptides described herein are type III-E effector including a crRNA processing domain. In some embodiments, the Cas peptide described herein is Cas7.1 of a Cas7-11 (e.g., Cas7.1 of DiCas7-11).

In some embodiments, the Cas peptides described herein are compact Cas peptides with smaller molecular size (about 100-1000 amino acids, e.g., about 100, about 120, about 140, about 160, about 180, about 200, about 220, about 230, about 238, about 250, about 260, about 280, about 300, about 350, about 400, about 450, about 500, about 550, about 560, about 570, about 580, about 582, about 600, about 650, about 700, about 750, about 800, about 850, about 900, about 950, or about 1000 amino acids) compared to wild-type Cas7-11 (1601 amino acids). In some embodiments, the Cas peptides described herein are truncated Cas7-11 (e.g., wild-type or with any mutations) or fragment thereof. In some embodiments, the Cas peptides described herein further include a target coordination domain (e.g., Cas 11), one or more (e.g., 1, 2, 3, or 4) nucleus localization sequence (NLS). In some embodiments, the Cas peptide optionally includes one or more (e.g., 1, 2, 3, 4, 5, 6, 7, 8, 9, or 10) additional domains (e.g., target coordination domain, enzymatic cleavage domain, or NLS), wherein the domains are linked by one or more (e.g., 1, 2, 3, 4, 5, 6, 7, 8, 9, or 10) linker sequences.

Examples of the Cas peptides described herein include, but are not limited to, a variant of Cas7-11, Cas 12, or Cas 13 without enzymatic cleavage activity. In some embodiments, the Cas peptide is inactivated Cas 7-11 (dCas7-11) with inactivated cleavage domains, dCas7.2 and/or dCas7.3. In some embodiments, dCas7.2 has a substitution of aspartic acid (D) by alanine (A) at position 429. In some embodiments, dCas7.3 has a substitution of aspartic acid (D) by alanine (A) at position 654. In some embodiments, the Cas peptides is a variant of dCas7-11 (e.g., Cas7.1, Cas7.1-dCas7.2, Cas7.1-dCas7.2-dCas7.3, Cas7.1-dCas7.2-dCas7.3-Cas7.4, Cas7.1-dCas7.3, Cas7.1-dCas7.3-Cas7.4, Cas7.1-Cas7.4, Cas7.1-Cas11, Cas7.1-Cas11-dCas7.2, Cas7.1-Cas11-dCas7.2-dCas7.3, Cas7.1-Cas11-dCas7.2-dCas7.3-Cas7.4, Cas7.1-Cas11-dCas7.3, Cas7.1-Cas11-dCas7.3-Cas7.4, or Cas7.1-Cas11-Cas7.4). In some embodiments, the Cas peptide described herein has an amino acid sequence at least 80%, 85%, 90%, 95%, 99%, or 100% identical to SEQ ID No.: 12 (coding sequence inserted in the plasmid of Addgene #220960) or SEQ ID No.:13 (coding sequence inserted in the plasmid of Addgene #220961).

As used herein, the Cas7-11 can be DiCas7-11, Cbf Cas7-11, CjcCas7-11, CmaCas7-11, CsbCas7-11, DpbaCas7-11, FmCas7-11, GwCas7-11, HreCas7-11, HsmCas7-11, HvmCas7-11, OmCas7-11, SmCas7-11, SstCas7-11, or SybCas7-11. In some embodiments, the Cas7-11 is DiCas7-11. In some embodiments, the Cas7-11 is DiCas7-11. In some embodiments, the Cas7-11 used herein is inactivated DiCas7-11 (dDiCas7-11) with inactivated cleavage domains, dCas7.2 and/or dCas7.3, wherein the dCas7.2 has a substitution of aspartic acid (D) by alanine (A) at position 429, and the dCas7.3 has a substitution of aspartic acid (D) by alanine (A) at position 654.

The crRNA processing domains described herein can process the crRNA to become a mature crRNA. In some embodiments, the crRNA processing domain is Cas7.1 (e.g., DiCas7.1, Cbf Cas7.1, CjcCas7.1, CmaCas7.1, CsbCas7.1, DpbaCas7.1, FmCas7.1, GwCas7.1, HreCas7.1, HsmCas7.1, HvmCas7.1, OmCas7.1, SmCas7.1, SstCas7.1, or SybCas7.1). In some embodiments, the crRNA processing domain is DiCas7.1 (SEQ ID No.: 12; coding sequence inserted in the plasmid of Addgene #220960). In some embodiments, the crRNA processing domain is at least a portion of Cas 6, Cas 12, or Cas 13.

### Gene Delivery Vectors

In some embodiments of any of the methods described herein, introducing a target sequence or the nucleic acid sequence encoding crRNA into the subject is facilitated by a vector (e.g., AAV).

For example, a vector can be an expression vector where the expression vector includes a promoter sequence operably linked to the sequence encoding the molecule (e.g., a nucleic acid molecule). Non-limiting examples of vectors include plasmids, transposons (e.g., DNA transposons, RNA transposons or retrotransposons, and class III transposons), cosmids, and viral derived vectors (e.g., any adenoviral derived vectors (AV), cytomegaloviral derived (CMV) vectors, simian viral derived (SV40) vectors, adeno-associated virus (AAV) vectors, lentivirus vectors, retroviral vectors, or any Gateway® vectors). A vector can, for example, include sufficient cis-acting elements for expression where other elements for expression can be supplied by the host mammalian cell or in an in vitro expression system. Skilled practitioners will be capable of selecting suitable vectors and mammalian cells for introducing any of spatial profiling reagents described herein.

### AAV Gene Delivery

Provided herein are methods of regulating expression of a gene provided by an adeno-associated virus (AAV) vector, including delivering, to a subject, a recombinant CRISPR-Cas system. In some embodiments, the AAV vector include, but is not limited to, AAV1, AAV2, AAV4, AAV5, AAV6, AV6.2, AAV7, AAV8, AAV9, rh.10, rh.39, rh.43, or CSp3. In some embodiments, the subject can be a mammal, a human, a rodent, a mouse, a rat, a cell, or a microbial cell. In some embodiments, the crRNA processing domain of the type III-E effector can be Cas7.1 of a Cas7-11. As described herein, the one or more DR is or are positioned upstream, downstream, or both upstream and downstream of the nucleotide sequence coding the gene.

In some embodiments of any of the methods described herein, the methods can be used to control or regulate the gene delivery strategies by AAV and variants thereof. As used herein, AAV is a preferred viral vector system useful for delivery of nucleic acids in the present methods. AAV is a tiny non-enveloped virus having a 25 nm capsid. No disease is known or has been shown to be associated with the wild type virus. AAV has a single-stranded DNA (ssDNA) genome. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. In some embodiments, the AAV is AAV1, AAV2, AAV4, AAV5, AAV6, AV6.2, AAV7, AAV8, AAV9, rh.10, rh.39, rh.43 or CSp3. In some embodiments, the AAV is chosen based on its natural tropism (see, e.g., Table 3).

TABLE 3
Characterization of AAV natural serotypes.

| Serotype | Natural Tropism |
|---|---|
| AAV1 | Muscle, CNS, heart, liver, lungs |
| AAV2 | Heart, CNS, liver, lungs, retina |
| AAV3 | Liver |
| AAV4 | Retina, lungs, kidney |
| AAV5 | Retina, CNS, liver |
| AAV6 | Heart, liver, muscle, retina |
| AAV7 | Liver |
| AAV8 | Muscle, heart, CNS, liver |
| AAV9 | Heart, CNS, liver |
| AAV10 | Muscle, myoblast tissue |
| AAV11 | Muscle, myoblast tissue |
| AAV12 | Salivary glands, muscle |

(Table 3 adapted from Table 1 of Korneyenkov and Zamyatnin, Jr., Pharmaceutics 2021, 13(5), 750).

### Methods of Making

Provided herein are methods of making the recombinant CRISPR-Cas system described herein including (a) synthesize a nucleic acid sequence encoding CRISPR RNA (crRNA) including a nucleotide sequence encoding the target sequence and one or more direct repeats (DRs); and (b) synthesize a nucleic acid comprising a nucleotide sequence encoding a Cas peptide or synthesize a recombinant Cas peptide using chemical and/or enzymatic reactions, wherein the recombinant Cas peptide comprises a crRNA processing domain. The CRISPR RNA (crRNA), a nucleic acid including a nucleotide sequence encoding a Cas peptide, and a recombinant Cas peptide described herein can be synthesized by any methods know in the art (e.g., chemical and enzymatic methods).

In some embodiments of the methods provided herein, the methods of making the recombinant CRISPR-Cas system further include purifying the synthesized nucleic acid sequence encoding crRNA. In some embodiments of the method provide herein, the methods of making the recombinant CRISPR-Cas system further include purifying the synthesized nucleic acid comprising a nucleotide sequence encoding a Cas peptide or purifying the synthesized Cas peptide. The methods can be any purifying methods known in the art.

### Methods of Gene Expression Regulation

Provided herein are methods of regulating a target sequence expression in a subject, and the methods include (a) identifying the target sequence; and (b) delivering, to the subject, a recombinant CRISPR-Cas system including a nucleic acid sequence encoding CRISPR-RNA (crRNA) including one or more direct repeats and a nucleotide sequence coding the target sequence; and a type III-E effector comprising a crRNA processing domain, wherein the type III-E effector and the nucleic acid sequence encoding crRNA are delivered at a ratio of about 1:5 to about 50:1. For example, the type III-E effector and the nucleic acid sequence encoding crRNA can be delivered at a ratio of about 20:1, 18:1, 16:1, 15:1, 13:1, 11:1, 10:1, 9:1, 8:1, 7:1, 6:1, 5:1, 4:1, 3:1 2:1, 1:1, or 1:2.

The methods described herein are capable to identify the minimal DR sequence required for robust gene knockdown, utilizing synthetic tools to generate multiple permutations of crRNA direct repeats (DRs) into mRNA molecules. The methods described herein are capable of both transcriptional and translational repression and can regulate the downstream activity of enzymes such as CRISPR-Cas9. Additionally, titration of CTRL effectors as used in the methods as disclosed herein leads to high effectiveness, facilitating tunable gene knockdown and making it a powerful tool for modeling variable gene expression in diverse applicable settings (e.g., bioreactors, AAV control strategies, viral quantification, drug/toxin regulation, or the Release of Insects Carrying a Dominant Lethal Gene (RIDL)).

The methods described herein can reduce the target sequence expression at mRNA and/or protein level. The methods described herein reduce the target sequence expression by endogenous machineries (e.g., exonuclease enzymes and/or exosomes) in the subject, thereby inducing less toxic or adverse effects to the subject as compared with other gene expression that is not regulated by endogenous exonuclease enzymes and/or exosomes. In some embodiments, the target sequences are endogenous sequences. In some embodiments, the target sequences are exogenous sequences. In some embodiments, the target sequences are coding sequences for proteins. In some embodiments, the target sequences non-coding sequence for non-coding RNA molecules (e.g., messenger RNA (mRNA), synthetic RNA, single-stranded RNA, double-stranded RNA, non-coding RNA, small RNA, or microRNA). The target sequences can code any genes known in art. The target sequences can code any synthetic genes or transgene described herein. In some embodiments, the target sequence codes genes, e.g., Cas 9 gene (e.g., SpyCas9-NG) or CasRx gene (e.g., RfxCas13d (CasRx)), that have regulatory functions on downstream genes, wherein the downstream genes can be permanently or transiently suppressed. In some embodiments, the methods described herein can reduce SpyCas9-NG indel formation by about 20%-60% (e.g., about 30%-50%, about 30%, about 40%, or about 48%). In some embodiments, the methods described herein can reduce SpyCas9-NG mRNA by at least 50% (e.g., at least 60%, at least 65%, at least 70%, at least 75%, at least 80%, at least 90%, at least 95%, about 60%-65%, about 60%-70%, about 60%-75%, about 60%-80%, about 60%-85%, about 60%-90%, about 60%, about 65%, or 80%). In some embodiments, the methods described herein can regulate transient CasRx-mediated gene expression (e.g., EGFP). In some embodiments, the methods described herein can restore EGFP expression by targeting CasRx. In some embodiments, EGFP mRNA expression is increased by about 1.2 to 5 fold (e.g., about 1.5, about 1.6, about 1.7, about 1.8, about 1.9, about 2.0, about 2.2, about 2.5, about 2.8, about 3.0, about 3.2, about 3.5, about 3.8, about 4.0, about 4.2, about 4.5, about 4.8, or about 5.0 fold).

In some embodiments, the target sequences can code a toxin gene, e.g., bacterial toxins, diphtheria toxin, botulinum toxin, ricin, cholera toxin, pertussis toxin, Bacillus thuringiensis (Bt) toxin, or aflatoxins.

In some embodiments, delivering the recombinant CRISPR-Cas system can be delivering a composition comprising one or more nucleic acids encoding the recombinant CRISPR-Cas system or fragments thereof. In some embodiments, delivering the recombinant CRISPR-Cas system can be delivering a composition comprising a first nucleic acid (e.g., polynucleotide) encoding the crRNA and a second nucleic acid (e.g., polynucleotide) encoding the type III-E effector. In some embodiments, delivering the recombinant CRISPR-Cas system comprises delivering a nucleic acid can be a polynucleotide encoding the crRNA and an amino acid sequence comprising the type III-E effector.

Employment of the methods described herein can reduce the expression level of the protein, encoded by the target sequence, by at least 40% (e.g., about 40%-100%, about 50%-100%, about 60%-100%, about 70%-100%, about 80%-100%, about 90%-100%, about 40%, about 45%, about 50%, about 55%, about 60%, about 65%, about 70%, about 75%, about 80%, about 85%, about 90%, about 95%, about 99%, or about 100%) or the expression level of RNA, encoded by the target sequence, by at least 40% (e.g., about 40%-100%, about 50%-100%, about 60%-100%, about 70%-100%, about 80%-100%, about 90%-100%, about 40%, about 45%, about 50%, about 55%, about 60%, about 65%, about 70%, about 75%, about 80%, about 85%, about 90%, about 95%, about 99%, or about 100%) compared to the protein level or RNA level without treatment the recombinant CRISPR-Cas system. In some embodiments, the methods described herein can reduce the protein level by at least 40% (e.g., about 40%-100%, about 50%-100%, about 60%-100%, about 70%-100%, about 80%-1000%, about 900%-100%, about 40%, about 45%, about 50%, about 55%, about 60%, about 65%, about 70%, about 75%, about 80%, about 85%, about 90%, about 95%, about 99%, or about 100%) and the RNA level by at least 40% (e.g., about 40%-100%, about 50%-100%, about 60%-100%, about 70%-100%, about 80%-100%, about 90%-100%, about 40%, about 45%, about 50%, about 55%, about 60%, about 65%, about 70%, about 75%, about 80%, about 85%, about 90%, about 95%, about 99%, or about 100%), wherein the protein and RNA are encoded by the target sequence. In some embodiments, the methods described herein can reduce the protein level by at least 70% (e.g., about 70%-100%, about 75%-100%, about 80%-100%, about 85%-100%, about 90%-100%, about 95%-100%, about 70%-80%, about 75%-95%, about 80%-90%, about 70%, about 75%, about 80%, about 85%, about 90%, about 95%, about 99%, or about 100%), while the RNA level was reduced by at least 80% (e.g., about 80%-100%, about 85%-100%, about 90%-100%, about 95%-100%, about 85%-95%, about 80%-90%, about 80%, about 85%, about 90%, about 95%, about 99%, or about 100%, wherein the protein and RNA are encoded by the target sequence. In some embodiments, the methods described herein can reduce the protein level by at least 70% (e.g., about 70%-100%, about 75%-100%, about 80%-100%, about 85%-100%, about 90%-100%, about 95%-100%, about 70%-80%, about 75%-95%, about 80%-90%, about 70%, about 75%, about 80%, about 85%, about 90%, about 95%, about 99%, or about 100%), while the RNA level was reduced less than 80% (e.g., about 0-79.9%, about 0-70%, about 0-60%, about 0-50%, about 0-40%, about 0-30%, about 0-20%, about 0-10%, about 0-5%, about 10%-70%, about 20%-60%, about 30%-50%, about 40%-70%, about 0, about 1%, about 5%, about 10%, about 15%, about 20%, about 25%, about 30%, about 35%, about 40%, about 45%, about 50%, about 55%, about 60%, about 65%, about 70%, or about 75%), wherein the protein and RNA are encoded by the target sequence.

### Regulation of Toxin Gene Expression in an Insect

Provided herein are methods of regulating a toxin gene expression in an insect (e.g., RIDL), and the methods include delivering, to the insect, a recombinant CRISPR-Cas system including a nucleic acid sequence encoding CRISPR-RNA (crRNA) and a type III-E effector comprising a crRNA processing domain. The type III-E effector and the nucleic acid sequence encoding crRNA are delivered at a ratio of about 1:5 to about 50:1. For example, the type III-E effector and the nucleic acid sequence encoding crRNA can be delivered at a ratio of about 20:1, 18:1, 16:1, 15:1, 13:1, 11:1, 10:1, 9:1, 8:1, 7:1, 6:1, 5:1, 4:1, 3:12:1, 1:1, or 1:2. The crRNA described herein includes one or more direct repeats and a nucleotide sequence coding the toxin gene, wherein the toxin gene is lethal or nonlethal. The type III-E effector described herein includes a crRNA processing domain. In some embodiments, the crRNA processing domain of the type III-E effector can be Cas7.1 of a Cas7-11. As described herein, one or more DR(s) can be positioned upstream, downstream, or both upstream and downstream of the nucleotide sequence coding the toxin gene. Also described herein are toxin genes including, but not limited to, bacterial toxins, diphtheria toxin, botulinum toxin, ricin, cholera toxin, pertussis toxin, Bacillus thuringiensis toxin, or aflatoxins. In some embodiments, the toxin genes are dominant lethal genes. The insect includes, but is not limited to, a fruit fly, a mosquito, bedbug, a housefly, a clothes moth, a Japanese beetle, an aphid, a flea, a horsefly, a hornet, a butterfly, or a moth.

The methods described herein can be used to control the Release of Insects carrying a Dominant Lethal gene (RIDL) system. RIDL is a genetic strategy used for pest control. Insects are engineered to carry a dominant lethal gene. When these genetically modified insects are released into the wild and mate with wild insects, the offspring inherit the lethal gene, causing death in offspring. The methods disclosed herein are genetic regulation methods that can repress the expression of the lethal gene at desired levels by modulating the ratio of the type III-E effector and the nucleic acid sequence encoding crRNA as described herein. Therefore, the methods disclosed herein control populations of insects (e.g., disease-carrying mosquitoes).

### Regulation of Gene Expression in a Bioreactor

Provided herein are methods of regulating expression of a gene in a culture container, the method including delivering, to a cell, a recombinant CRISPR-Cas system. The cells used herein include, but are not limited to, mammalian cells, microbial cells, cell lines, primary cells, or transgenic cells. In some embodiments, the crRNA processing domain of the type III-E effector can be a comprising Cas7.1 of a Cas7-11. In some embodiments of the methods described herein, the one or more DR can be positioned upstream, downstream, or both upstream and downstream of the nucleotide sequence coding the gene.

Bioreactors are used to cultivate cells or microorganisms under optimal conditions to achieve high levels of gene expression. In some embodiments of the methods described herein, the methods of delivering a recombinant CRISPR-Cas system to the cells cultivated in bioreactors can control gene expression level by modulating the ratio of the type III-E effector and the nucleic acid sequence encoding crRNA as described herein. Bioreactor technology is widely used in pharmaceuticals, biotechnology, and research for producing vaccines, therapeutic proteins, and other biologics. Bioreactors allow for the scale-up of production from small laboratory settings to industrial levels, making it possible to produce large quantities of the desired product. Various culture volumes can be employed in bioreactors, ranging from about 500 ml to over 10,000 liters (L) (e.g., about 500 ml, about 1000 ml, about 2000 ml, about 3000 ml, about 4000 ml, about 5 L, about 6 L, about 7 L, about 8 L, about 9 L, about 10 L, about 20 L, about 50 L, about 80 L, about 100 L, about 200 L, about 300 L, about 400 L, about 500 L, about 600 L, about 700 L, about 800 L, about 900 L, about 1000 L, about 2000 L, about 3000 L, about 4000 L, about 5000 L, about 6000 L, about 7000 L, about 8000 L, about 9000 L, about 10,000 L, or over 10,000 L.

### Subjects

The term “subject” is used interchangeably throughout the specification and describe an animal or a cell. In some embodiments, the methods described herein involves using the subject as recipient of the CTRL system described herein, and the subject express the target sequence as described herein (e.g., a reporter gene, a functional gene, a synthetic gene, or a transgene. Veterinary and non-veterinary applications are contemplated by the present invention. Examples of subjects include, but are not limited to, human, mice, rats, hamsters, guinea-pigs, rabbits, ferrets, cats, dogs, primates, or insects. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals. In some embodiments, an insect can include a bedbug, a housefly, a clothes moth, a Japanese beetle, an aphid, a mosquito, a flea, a horsefly, a hornet, a butterfly, a fruitfly, or a moth. In some embodiments, an insect can be a mosquito from the genera Stegomyia, Aedes, Anopheles, or Culex. In some embodiments, the mosquito can include Aedes aegypti, Aedes albopictus, Ochlerotatus triseriatus (Aedes triseriatus), Anopheles stephensi, Anopheles albimanus, Anopheles gambiae, Anopheles quadrimaculatus, Anopheles freeborni, Culex species, or Culiseta melanura. In some embodiments, the insect can include a tephritid fruit fly selected from Medfly (Ceratitis capitata), Mexfly (Anastrepha ludens), Oriental fruit fly (Bactrocera dorsalis), Olive fruit fly (Bactrocera oleae), Melon fly (Bactrocera cucurbitae), Natal fruit fly (Ceratitis rosa), Cherry fruit fly (Rhagoletis cerasi), Queensland fruit fly (Bactrocera tyroni), Peach fruit fly (Bactrocera zonata), Caribbean fruit fly (Anastrepha suspensa), Oriental Fruit Fly (Bactrocera dorsalis), West Indian fruit fly (Anastrepha obliqua), the New World screwworm (Cochliomyia hominivorax), the Old World screwworm (Chrysomya bezziana), Australian sheep blowfly/greenbottle fly (Lucilia cuprina), the pink bollworm (Pectinophora gossypiella), the European Gypsy moth (Lymantria dispar), the Navel Orange Worm (Amyelois transitella), the Peach Twig Borer (Anarsia lineatella), the rice stem borer (Tryporyza incertulas), the noctuid moths, Heliothinae, the Japanese beetle (Papilla japonica), White-fringed beetle (Graphognatus spp.), Boll weevil (Anthonomous grandis), the Colorado potato beetle (Leptinotarsa decern lineata), the vine mealybug (Planococcus ficus), Asian citrus psyllid (Diaphorina citri), Spotted wing drosophila (Drosophila suzukii), Bluegreen sharpshooter (Graphocephala atropunctata), Glassy winged sharpshooter (Flomalodisca vitripennis), Light brown apple moth (Epiphyas postvittana), Bagrada bug (Bagrada hilaris), Brown marmorated stink bug (Halyomorpha halys), Asian Gypsy Moth selected from the group of Lymantria dispar asiatica, Lymantria dispar japonica, Lymantria albescens, Lymantria umbrosa, and Lymantria postalba, Asian longhorned beetle (Anoplophora glabripennis), Coconut Rhinoceros Beetle (Oryctes rhinoceros), Emerald Ash Borer (Agrilus planipennis), European Grapevine Moth (Lobesia botrana), European Gypsy Moth (Lymantria dispar), False Codling Moth (Thaumatotibia leucotreta), fire ants selected from Solenopsis invicta Buren, and S. richteri Forel, Old World Bollworm (Flelicoverpa armigera), Spotted Lanternfly (Lycorma delicatula), Africanized honeybee (Apis mellifera scutellata), Fruit and shoot borer (Leucinodes orbonalis), corn root worm (Diabrotica spp.), Western corn rootworm (Diabrotica virgifera), Whitefly (Bemisia tabaci), Flouse Fly (Musca domestica), Green Bottle Fly (Lucilia cuprina), Silk Moth (Bombyx mori), Red Scale (Aonidiella aurantia), Dog heartworm (Dirofilaria immitis), Southern pine beetle (Dendroctonus frontalis), Avocado thrip (Thysanoptera spp.), Botfly selected from Oestridae spp. and Dermatobia hominis), Florse Fly (Tabanus sulcifrons), Florn Fly (Flaematobia irritans), Screwworm Fly selected from Cochliomyia macellaria (C. macellaria), C. hominivorax, C. aldrichi, or C. minima, Tsetse Fly (Glossina spp.), Warble Fly selected from Flypoderma bovis or Hypoderma lineatum, Spotted lanternfly (Lycorma delicatula), Khapra beetle (Trogoderma granarium), Honeybee mite (Varroa destructor), Termites (Coptotermes formosanus), Hemlock woolly adelgid (Adelges tsugae), Walnut twig beetle (Pityophthorus juglandis), European wood wasp (Sirex noctilio), Pink-spotted bollworm (Pectinophora scutigera), Two spotted spider mite (Tertanychus urticae), Diamondback moth (Plutella xylostella), Taro caterpillar (Spodoptera litura), Red flour beetle (Tribolium castaneum), Green peach aphid (Myzus persicae), Cotton Aphid (Aphis gossypii), Brown planthopper (Nilaparvata lugens), Beet armyworm (Spodotera exigua), Western flower thrips (Frankliniella occidentalis), Codling moth (Cydia pomonella), Cowpea weevil (Callosobruchus maculatus), Pea aphid (Acyrthosiphon pisum), Tomato leafminer (Tuta absoluta), Onion thrips (Thrips tabaci), or Cotton bollworm (Helicoverpa armigera).

The cell used herein refers to any type of primary cells or cell lines that have been described in the art. Examples of cells include, but are not limited to mammalian cells, microbial cells, cell lines (e.g., HEK293T cells), primary cells, or transgenic cells.

### Pharmaceutical Compositions and Methods of Administration

Provided herein are pharmaceutical compositions including the recombinant CRISPR-Cas system including (a) a nucleic acid sequence encoding CRISPR RNA (crRNA) including one or more direct repeats (DRs) and a polynucleotide sequence encoded by the synthetic gene or transgene; and (b) a Cas peptide including a crRNA processing domain, wherein the recombinant CRISPR-Cas system does not reduce endogenous gene expression.

Provided herein are pharmaceutical compositions including the recombinant CRISPR-Cas system including a nucleic acid sequence encoding CRISPR-RNA (crRNA) including one or more direct repeats and a nucleotide sequence coding the target sequence; and a type III-E effector including a crRNA processing domain.

Also provided herein are methods of using the pharmaceutical compositions described herein.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Pharmaceutical compositions are typically formulated to be compatible with its intended route of administration into subjects. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

### EXAMPLES

The materials and methods described here have been used to generate the examples described herein.

### Materials and Methods

### Construct Design and Cloning

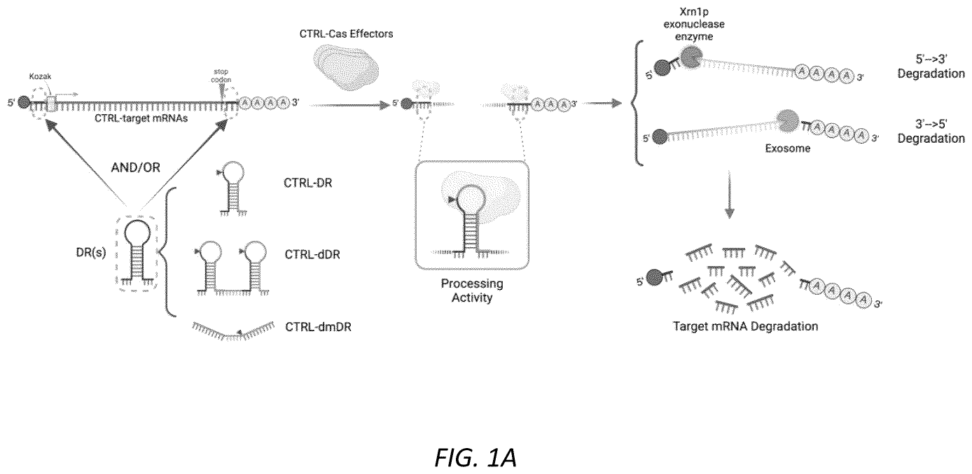

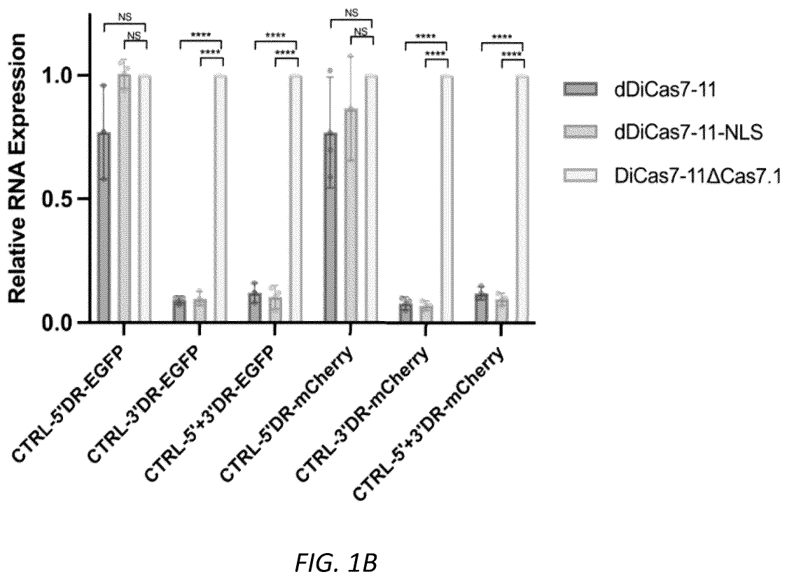

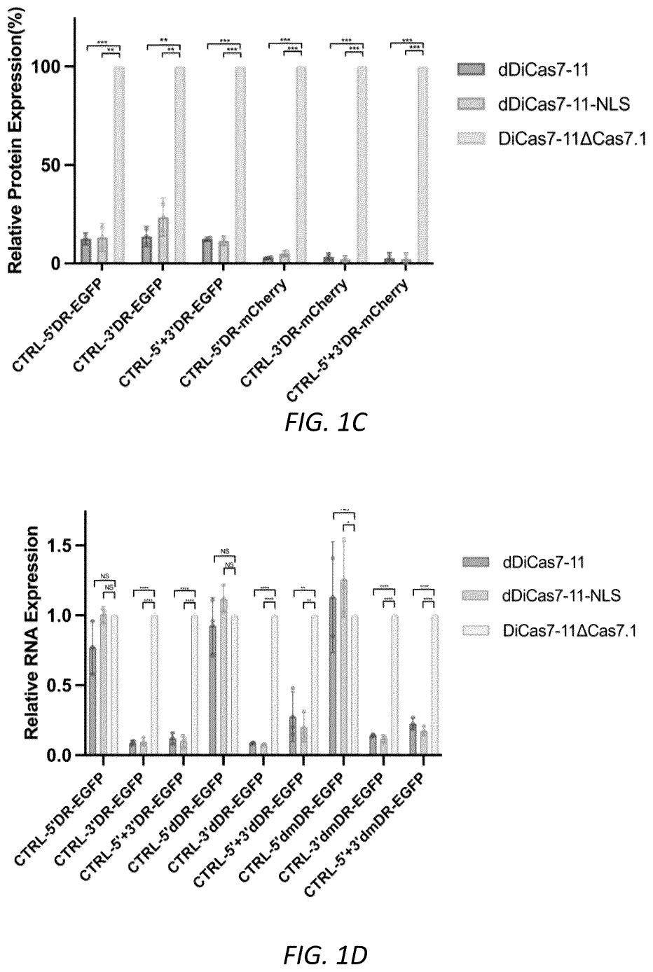

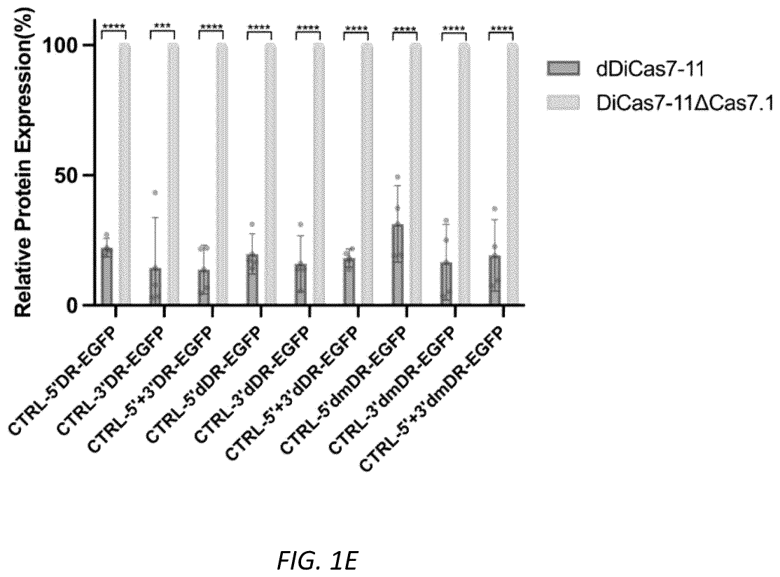

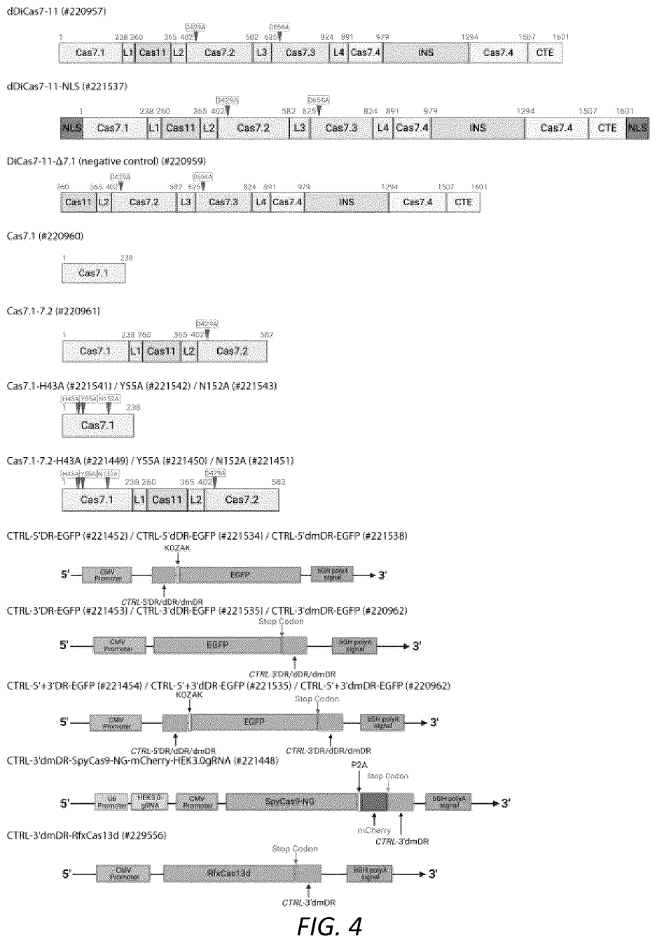

All plasmids were generated using the standard Gibson Assembly® methods. The reporter and effector expression plasmid were designed to contain the CMV promoter and the bovine growth hormone (bGH) terminator. The 5′ DRs were encoded downstream of the CMV promoter, but upstream of the Kozak sequence and start codon, whereas the 3′ DRs were encoded downstream of the stop codon (FIG. 1A and FIG. 4). The inactivated Cas7-11 constructs were generated by mutating the active residues in the Cas7.2 and Cas7.3 domains (D429A/D654A). All plasmids (FIG. 4) generated are shown in the table below and the sequences are available at Addgene.

TABLE 4
Plasmids generated in the examples

| Addgene | Plasmid |
|---|---|
| 220957 | 1203M-CMV-dDiCas7-11-bGHpolyA |
| 220959 | 1203O-CMV-DiCas7-11-Δ7.1-bGHpolyA |
| 220960 | 1203P-CMV-Cas7.1-bGHpolyA |
| 220961 | 1203W-CMV-Cas7.1-7.2-bGHpolyA |
| 220962 | 1203R-CMV-3′dmDR-EGFP-bGHpolyA |
| 221448 | 1203U-U6-gRNA-pCMV-wtCas9-NG-P2A-mcherry-3′dmDR |
| 221449 | 1203AE-CMV-Cas7.1-7.2-H43A-mutant |
| 221450 | 1203AF-CMV-Cas7.1-7.2-Y55A-mutant |
| 221451 | 1203AG-CMV-Cas7.1-7.2-N152A-mutant |
| 221452 | 1203A-CMV-5′DR-EGFP-bGHpolyA |
| 221453 | 1203B-CMV-3′DR-EGFP-bGHpolyA |
| 221454 | 1203C-CMV-5′ + 3′DR-EGFP-bGHpolyA |
| 221534 | 1203D-5′dDR-mCh(22)-GFP-bGHpolyA |
| 221535 | 1203E-3′dDR-mCh(22)-GFP-bGHpolyA |
| 221536 | 1203F-5′ + 3′dDR-mCh(22)-GFP-bGHpolyA |
| 221537 | 1203N-CMV-dDiCas7-11-NLS-bGHpolyA |
| 221538 | 1203Q-5′dmDR-NT(22)-GFP-bGHpolyA |
| 221539 | 1203S-5′ + 3′dmDR-NT(22)-GFP-bGHpolyA |
| 221540 | 1203T-CMV-Cas7.1-NLS-bGHpolyA |
| 221541 | 1203AB-CMV-Cas7.1-H43A-mutant |
| 221542 | 1203AC-CMV-Cas7.1-Y55A-mutant |
| 221543 | 1203AD-CMV-Cas7.1-N152A-mutant |
| 229556 | 3′dmDR-CMV-CasRx-bGHpolyA |

### Cell Culture Assays

All experiments used HEK293T cell lines obtained from the American Type Culture Collection (ATCC CRL-3216™). The cells were incubated at 37° C. and 5% CO₂. For all cell experiments except for the Cas9 repression assay, the HEK293T cells were cultured in Dulbecco's Modified Eagle Medium (DMEM) (Thermo Fisher Scientific 11995073) with 10% Fetal Bovine Serum (FBS) (Corning® 35-011-CV) and 1% penicillin-streptomycin (Thermo Fisher Scientific 15070063). 3.2×10⁴ cells were seeded in a 48-well dish approximately 22 hours before transfection, ensuring that their confluency was less than 60%. Transient transfections were carried out with Lipofectamine™ 3000 transfection reagent (Thermo Fisher Scientific L3000001) per the manufacturer's protocol. The HEK293T cells were co-transfected with 20 ng EGFP/mCherry CTRL-Reporter plasmids and the quantity of Cas7-11-based effector plasmids for the intended molar ratio. The cells were incubated for 48 hours before harvesting for experiments.

For the SpyCas9-NG expression assay, the cells were cultured in DMEM with 10% FBS, without antibiotics (penicillin-streptomycin). 5.0×10⁴ cells were seeded in a 48-well plate 24 hours before transfection, and the transient transfections were conducted with Lipofectamine™ 2000 transfection reagent (Thermo Fisher Scientific 11668030) based on the manufacturer's protocol. The cells were harvested 72 hours after transient transfection, and the culture media was changed once, 48 hours after the transfection.

qPCR and Data Analysis for RNA Expression

Cells were removed from the 48-well plate with 250 μL of RNAprotect® Cell Reagent (Qiagen 76104) 48 hours after transfection and stored at −20° C. for at least 24 hours prior to extraction. Cells were lysed using QIAshredder homogenizers (Qiagen 79656), and total RNA was then extracted using the RNeasy® Mini Kit (Qiagen 74106) based on the manufacturer's protocol. The total RNA was treated with TURBOT DNase (Thermo Fisher Scientific AM1907) to remove any remaining DNA. The concentration of the RNA samples was measured using a NanoDrop™ OneC microvolume UV-vis spectrophotometer (Thermo Fisher Scientific ND-ONE-W). Approximately 0.75 g of total RNA was used to synthesize cDNA with a Thermo Scientific™ RevertAid First Strand cDNA Synthesis Kit (Thermo Fisher Scientific K1622). cDNA was diluted (1:1000) for qPCR analysis. qPCR was performed with SYBR green qPCR master mix (qPCRBIO SyGreen Blue Mix Separate-ROX 17-507B, Genesee Scientific), using 4 μl of dilute cDNA in each 20 μl reaction containing a final primer concentration of 200 nM and 10 μl of SYBR green buffer solution. Three technical replicates were performed for each reaction. Pipetting was performed using epMotion® 5075 (Eppendorf). The following qPCR protocol was used on the LightCycler® 96 instrument (Roche): 3 min of activation phase at 95° C., 40 cycles of 5 sec at 95° C., and 30 sec at 60° C.

The relative EGFP expression was calculated using the manufacturer's software and the delta-delta Ct method (2^{-ΔΔCt}), using GAPDH as the reference gene. To assess differences in the EGFP RNA expression between the treatments with CTRL-effectors and the negative-control effector, statistical analysis was performed using GraphPad Prism 10. Specifically, a one-way ANOVA test was used, followed by the posthoc analysis with a Dunnett's multiple comparisons test and/or a Tukey's multiple comparisons test.

### Flow Cytometry and Data Analysis for Protein Repression Assessment

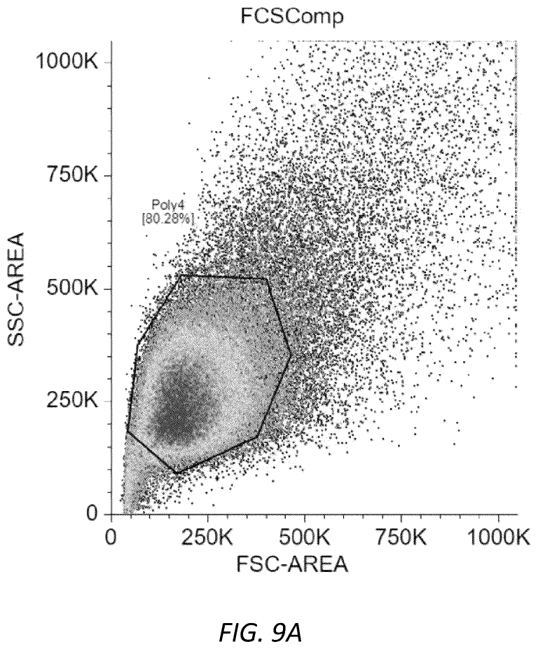

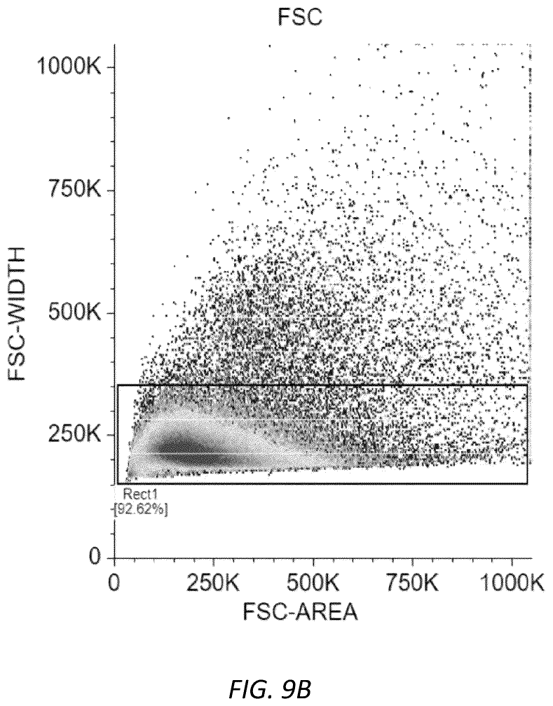

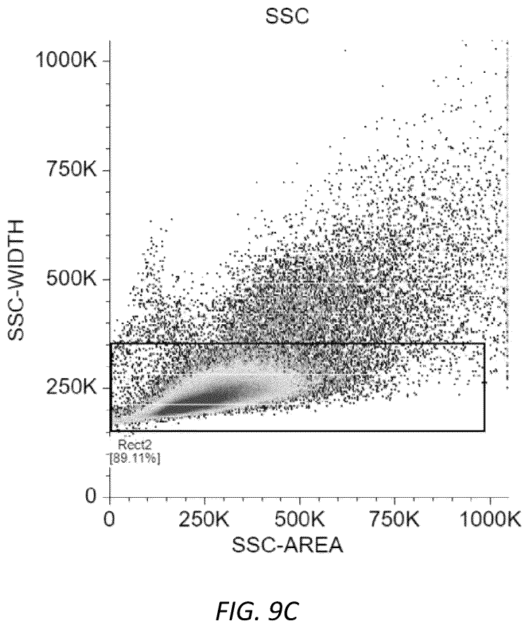

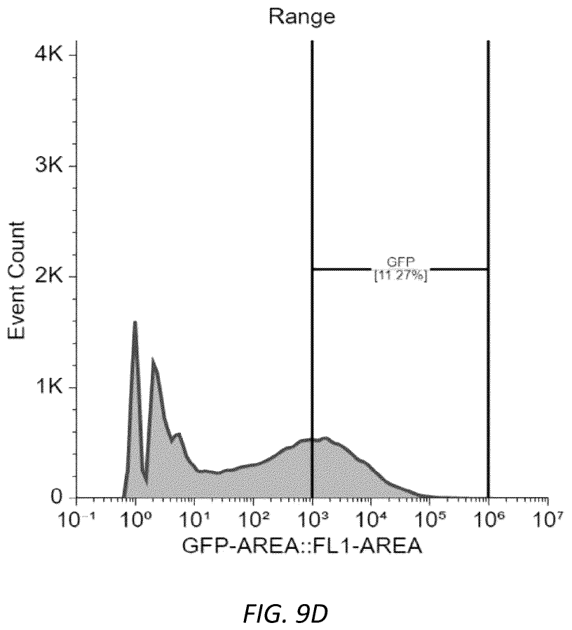

Fluorescence knockdown was assessed using flow cytometry on the Bio-Rad S3e cell sorter system. To prepare cells for analysis, transfected HEK293T cells were washed with 250 ul PBS pH 7.4 (Gibco 10010031), all media was removed, and then 40 μL of Accumax™ cell dissociation solution (VWR AM105) was added to the wells. 227 μL of cold PBS (pH 7.4) was added to the wells, and cells were resuspended before being transferred to a Falcon® cell strainer (Corning 352235). Cells were then assessed on the S3e cell sorter with a count of 80,000 cells per sample. Flow cytometry data were analyzed using Floreada.io (floreada.io/) (FIG. 9).

### Library Preparation for High-Throughput Sequencing and Data Analysis

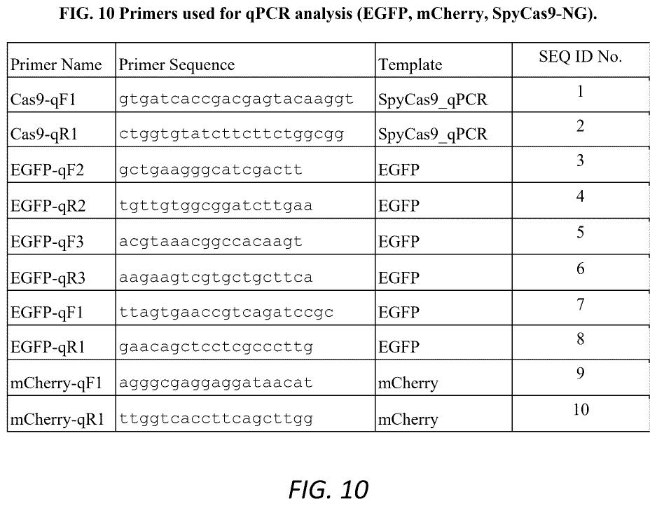

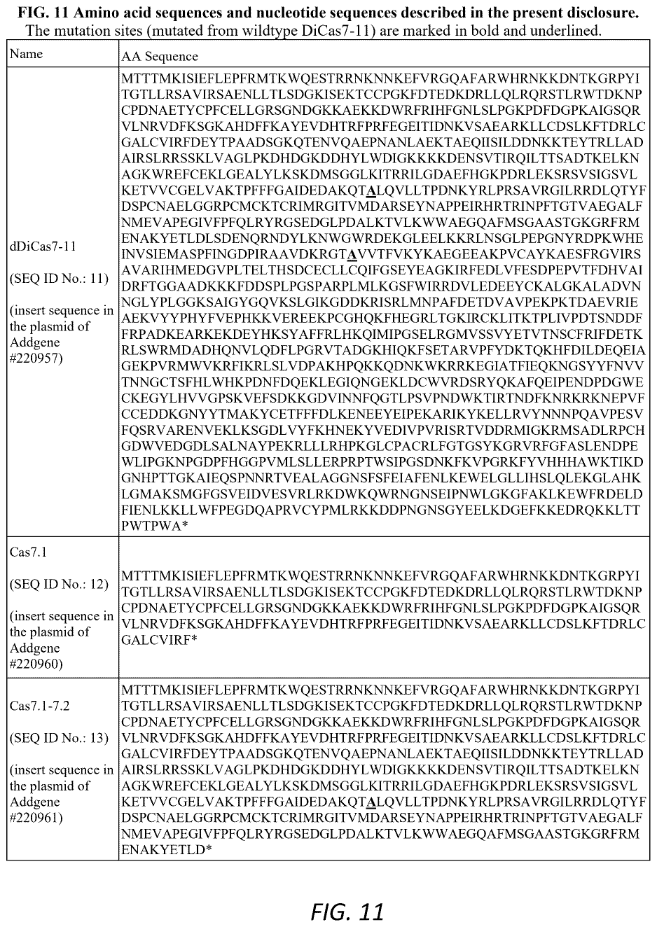

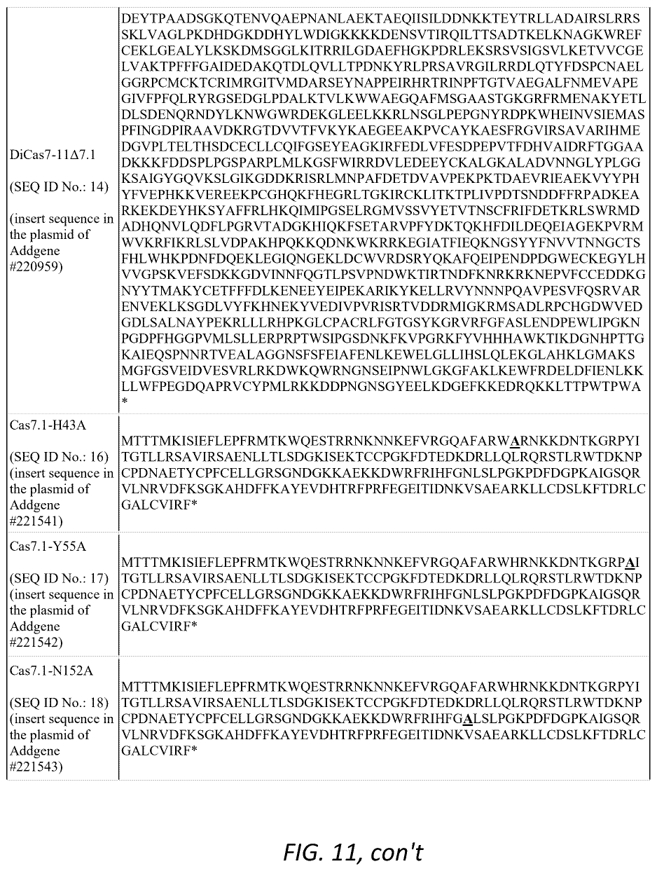

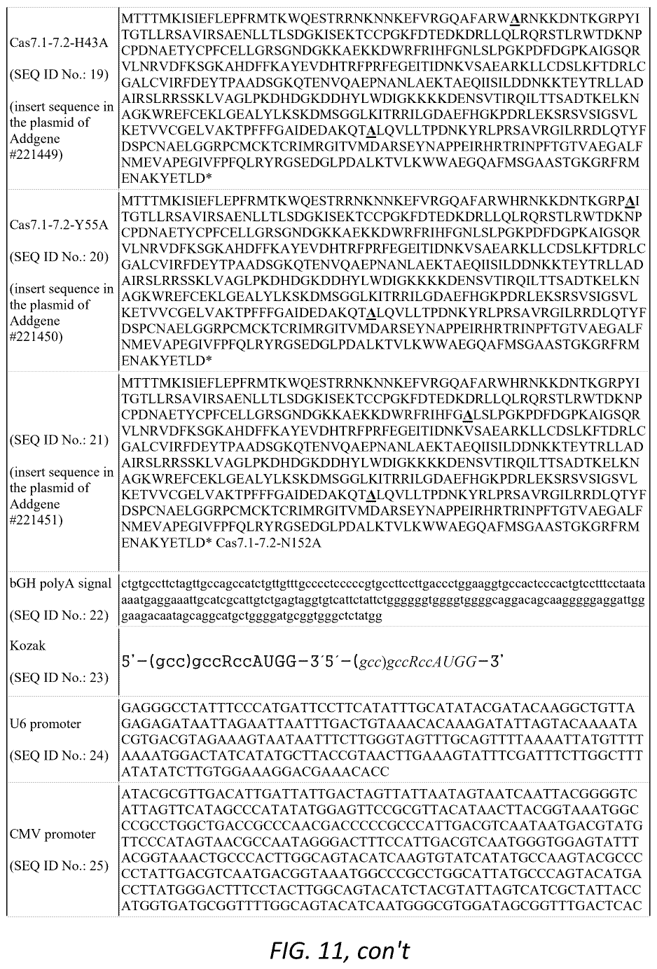

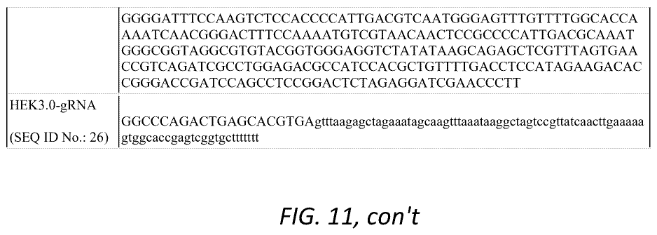

The plasmids encoding the CTRL-effectors (dDiCas7-11, Cas7.1, Cas7.1-7.2, DiCas7-11ΔCas7.1) and CTRL-3′dmDR-SpyCas9-NG with HEK293-site-3 sgRNA were transiently transfected into HEK293T cells at a molar ratio of 15:1. The media was removed 72 hours after transfection. The cells were washed with PBS and then lysed with the 100 ul freshly-made lysis buffer (at a 200 uL scale: 196.75 uL H2O, 2 uL Tris-EDTA, luL 1% SDS, 0.25 uL Proteinase K enzyme (Thermo Scientific™ E00491]) per confluent well (of a 48-well plate). The amount of lysis buffer was adjusted based on the cell confluency of each well (100 uL for fully confluent wells). The cells were incubated in the lysis buffer for 5 minutes, then resuspended and removed from the well into PCR tubes. The following thermocycler protocol was used for cell lysis: a) 1 hour at 37° C.; b) 30 minutes at 80° C.; c) ramp to 12° C. The lysed cells were stored at 4° C. The lysis (1-2 uL) was used as the template in the round 1 PCR and was amplified using high-throughput sequencing primers containing Illumina adapters (FIG. 10). Phusion® Hot Start Flex DNA Polymerase (New England Biolabs (NEB) M0535S) was used in the reactions, according to the manufacturer's instructions, with 27 cycles of amplification. The size and the relative concentration of each PCR sample were determined by gel electrophoresis. The round 1 PCR product (0.5-luL) was then amplified in a round 2 PCR for multiplexing with Illumina primers with unique barcodes and P5/P7 tail sequences. Round 2 PCR was completed using Phusion® High-Fidelity DNA Polymerase (NEB M0530L) for 12 cycles of amplification. After determining the relative concentration of each sample using gel electrophoresis, concentrations were normalized by pooling 1-10 uL of each round 2 PCR product and then purified via gel purification with the Monarch® Spin DNA Gel Extraction Kit (NEB T1020) based on the manufacturer's protocol. The purified samples were sequenced with a 300-cycle paired-end NGS run on a MiniSeq high-throughput DNA sequencer (Illumina).

Sequencing reads were demultiplexed using MiniSeq Reporter (Illumina), and FASTQ files were analyzed with CRISPResso2 software pipeline. CRISPRessoBatch analysis was used to analyze and compare multiple experimental conditions at the same site, which generated the indel frequency of each sample within the same quantification window (8-bp sequences that flank both sides of the predicted cleavage site).

### Availability of Data and Materials

Complete sequence maps and plasmids are deposited at Addgene.org (Catalog Numbers: #220957, #221537, #220959, #220960, #220961, #221541, #221449, #221450, #221451, #221452, #221534, #221538, #221453, #221535, #220962, #221448, and #229556 are available in FIG. 4).

### Example 1. CRISPR-Cas Transgenic Repressible eLement (CTRL) is Capable of Effectively Downregulating RNA and Protein Levels

To determine if CTRL can effectively downregulate protein expression, constructs were designed to include CRISPR-RNA direct repeats (crRNA DRs) encoded upstream and/or downstream of the target coding sequence for the gene of interest (FIG. 1A). Previous studies demonstrated that DiCas7-11 can efficiently process the 5′ direct repeats (DRs) of pre-crRNA molecules into mature crRNAs. DiCas7-11-based effectors were hypothesized to be able to process the DRs encoded in a transcribed construct and remove either the 5′ cap and/or the 3′ poly-A tail depending on the DR placement. To support strict crRNA processing and avoid non-specific cleavage, target cleavage for the Cas7-11 constructs were catalytically inactivated (FIG. 4). Additionally, since the Cas7.1 domain was responsible for crRNA processing in Cas7-11 proteins, a Cas7.1 deletion mutant (DiCas7-11ΔCas7.1) was created to serve as a negative control effector in this study (FIG. 4). It was hypothesized that, following DR processing, the targeted mRNAs would be degraded by the endogenous cell machinery, allowing for transient regulation of both mRNA and protein expression.

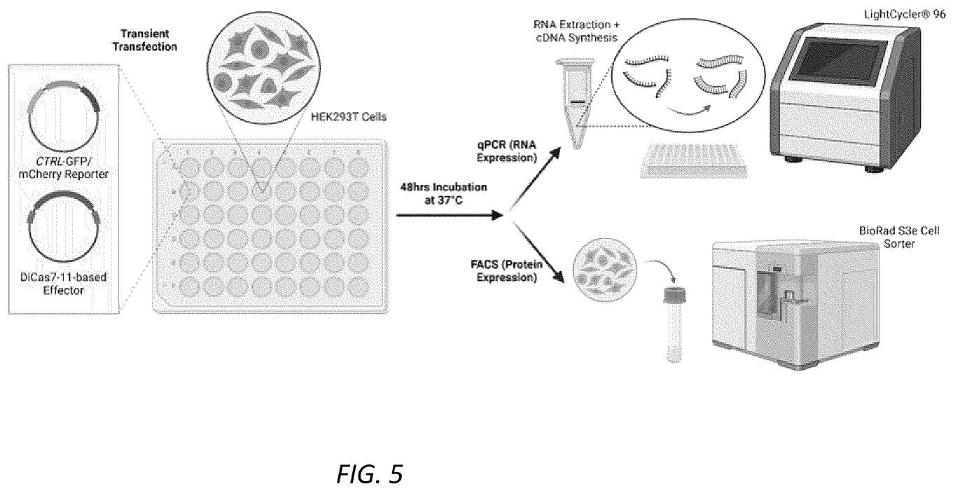

EGFP- and mCherry-reporter assays were developed to assess the efficacy of the CTRL system, evaluating mRNA and protein expression using RT-qPCR and flow cytometry, respectively (FIG. 5). The initial EGFP- and mCherry-reporter screens consisted of three different constructs encoding a single 35 nucleotide unprocessed direct repeats (CTRL-DRs) at the 5′, the 3′ end, or both the 5′ and 3′ ends of the EGFP or mCherry transcript (FIG. 4). In both reporter assays, with the inactivated DiCas7-11 (dDiCas7-11), over 90% reduction in RNA expression was observed in constructs with a DR encoded at the 3′ end or both the 3′ and 5′ ends of the transcript, while almost no knockdown was observed with a DR at the 5′ end only, as compared to the negative control effector DiCas7-11ΔCas7.1 (FIG. 1B). However, the flow cytometry data revealed that EGFP and mCherry protein expression was reduced by >80% in all constructs, including the 5′ encoded DR (FIG. 1C). It was also hypothesized that fusing a Nuclear Localization Signal (NLS) to the Cas7-11 protein would result in earlier RNA processing and, therefore, greater gene knockdown. However, the addition of an NLS did not significantly improve repression by CTRL (FIGS. 1B-1C).

### Example 2. CTRL has Flexible Design Criteria and is Effective at Low Molar Ratios

Next, the design strategy was expanded to improve and achieve consistent RNA and protein knockdown efficiency. Alternative DRs were explored in the EGFP-reporter constructs to generate constructs with double unprocessed DRs (CTRL-dDR) and double processed (or mature) DRs (CTRL-dmDR) (FIG. 1A and FIG. 4) at the 5′, 3′, or both the 5′ and 3′ ends of the transcript. Except for the constructs with DRs inserted at the 5′ end, 80% to 90% RNA knockdown was observed for all other constructs, with no significant differences in knockdown between designs (F(5, 12)=0.02110, P=0.9998) (FIG. 1D). However, there was a substantial reduction in EGFP protein levels for all constructs, including the constructs with 5′ encoded DRs (F (9, 30)=21.28, P<0.0001) (FIG. 1E).

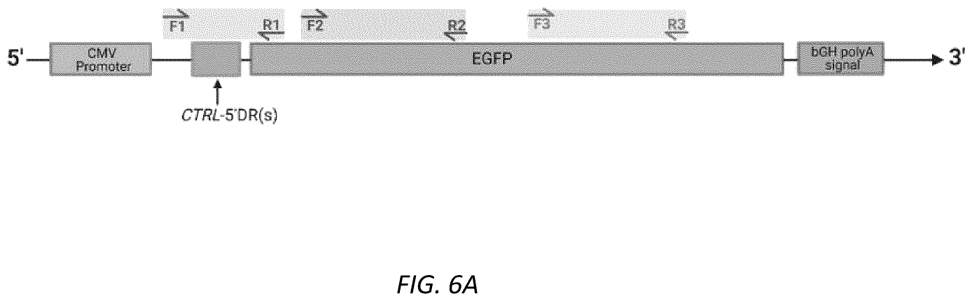

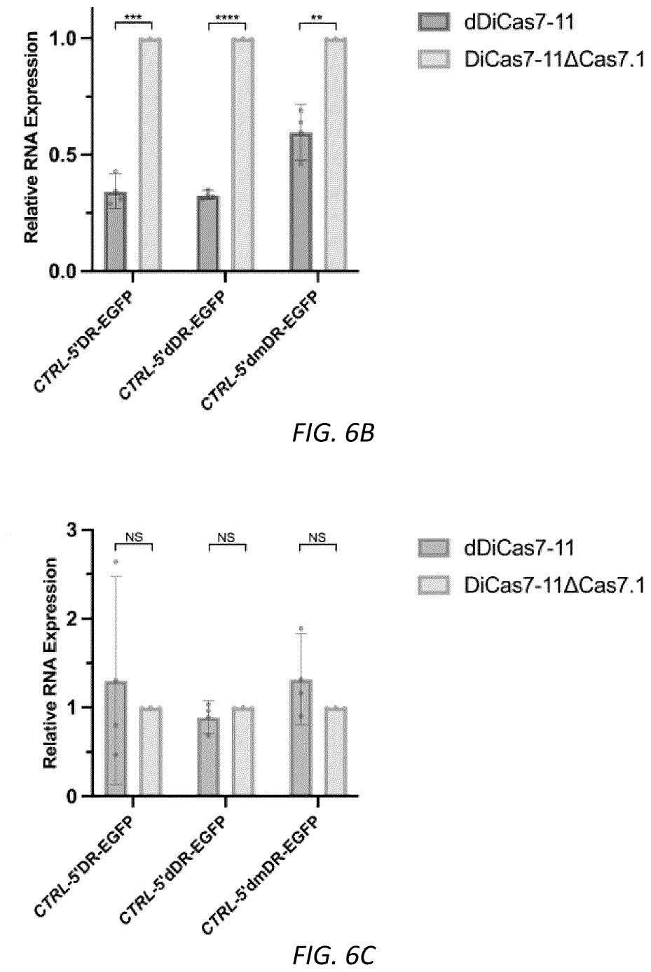

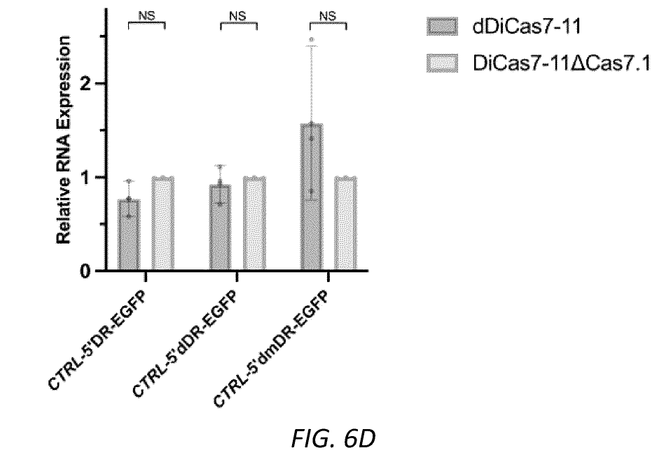

To reconcile the differences in protein and RNA knockdown with 5′ encoded DRs, an RT-qPCR analysis was performed with three primer pairs: F2-R2 and F3-R3, that detected transcripts within the EGFP-reporter coding sequence; and F1-R1, which amplifies around the DRs at the 5′ end of the transcript (FIG. 6A). The relative RNA expression in the EGFP coding region did not indicate RNA knockdown, but RNA knockdown was observed in the 5′ DR by 40-70% (FIGS. 6B-6D). These data suggested that the 5′ DRs were processed, but the transcripts were not degraded. Therefore, this approach reduced protein expression but did not result in the turnover of the targeted mRNA molecule.

The assays described above were conducted at an Effector-to-Reporter molar ratio (Effector:Reporter) of 15:1. To evaluate the protein knockdown capabilities at lower Effector:Reporter ratios, the effective ratio was lowered from 15:1 to 10:1 and conducted the same flow cytometry analysis for all 9 designs. The reduction in protein levels was found to decrease by about 10%, to 70-80% (while it was 80-90% when the molar ratio was 15:1). Again, no significant difference was observed in overall protein knockdown across all designs (FIG. 1E). Therefore, the 3′ CTRL-dmDR design was selected for all subsequent assays. This construct design, the 3′ CTRL-dmDR, is minimal, encodes two processing sites, and achieves consistent RNA and protein downregulation.

### Example 3. A C-Terminal Truncation of the Cas7-11 Protein Effectively Reduces Gene Expression

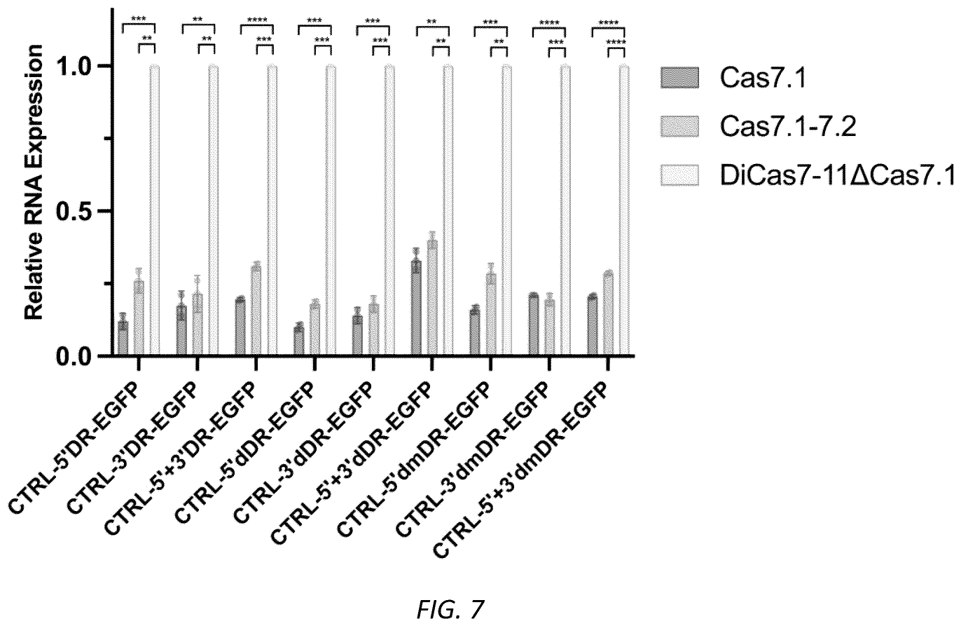

Two truncated DiCas7-11 proteins were created to test in the CTRL system: 1) a complete truncation from the C-terminus leaving only the Cas7.1 domain (Cas7.1), and 2) a partial C-terminal truncation with intact Cas7.1, and Cas11 domains, an inactive Cas7.2 domain, and the linkers in between the domains (Cas7.1-7.2) (FIG. 4). These truncated wild-type DiCas7-11 variants were incapable of crRNA-dependent target binding or cleavage and were, therefore, specific to DR processing. Both Cas7.1 and Cas7.1-7.2 significantly repressed mRNA expression of all 9 CTRL-DR(s)-EGFP constructs, indicating that the truncation did not abolish their processing activities (FIG. 7).

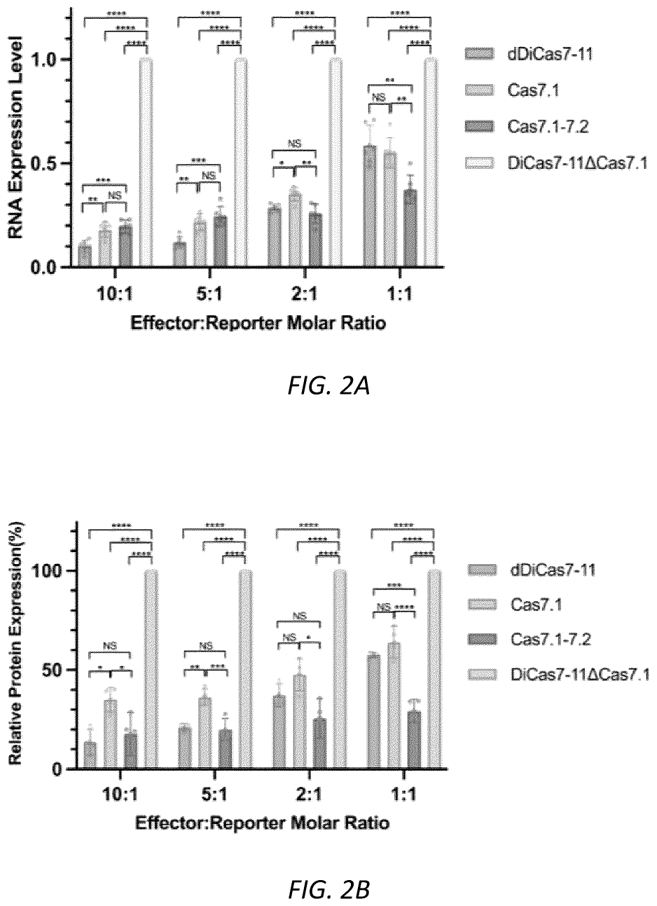

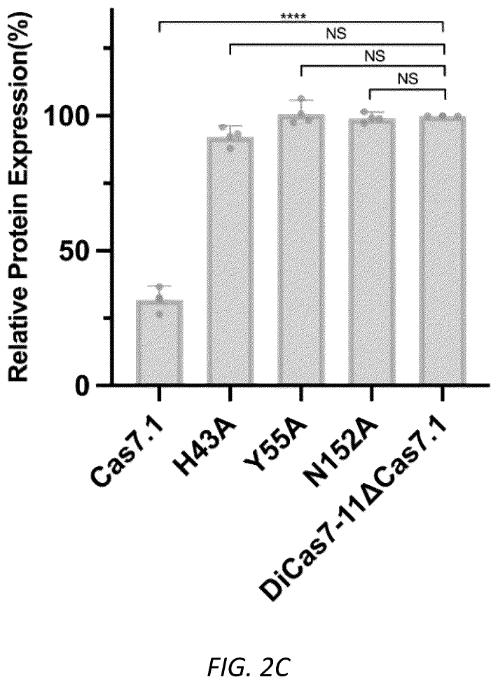

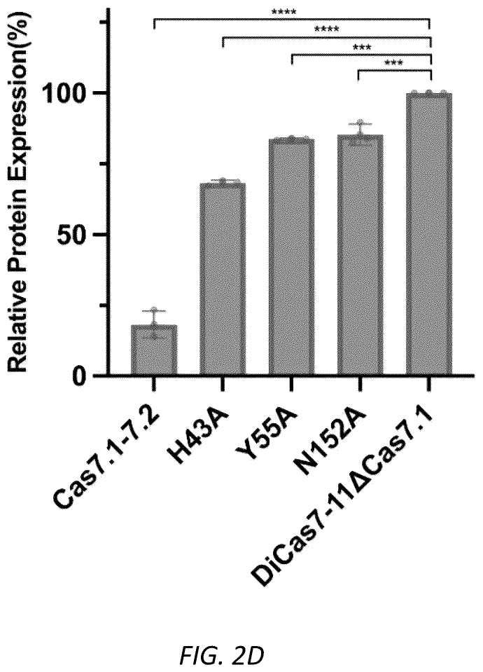

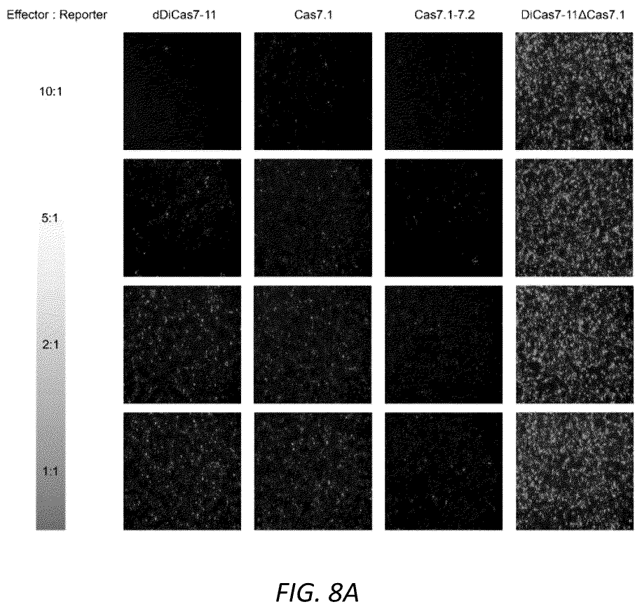

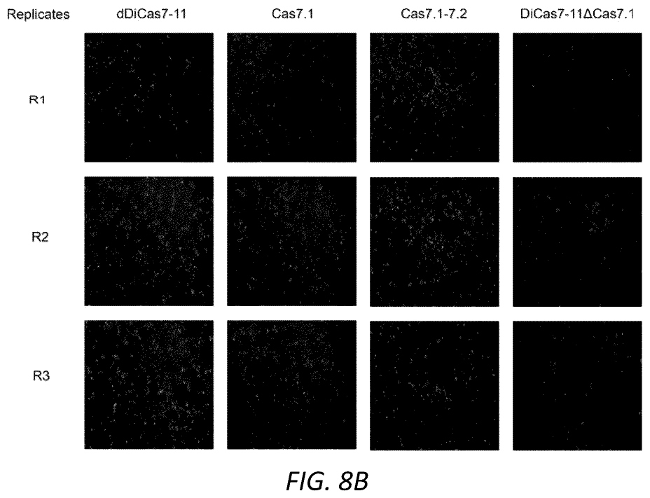

To distinguish the repression efficiencies of all CTRL-effectors, an Effector:Reporter molar ratio titration assay was performed to test the Effector:Reporter molar ratio from 1:1 to 10:1. A reduction in EGFP RNA and protein levels was observed for all truncated CTRL-effectors tested, with repression increasing with the Effector:Reporter ratio (FIGS. 2A-2B and FIG. 8A). At high molar ratios (10:1 and 5:1), the full-length dDiCas7-11 protein exhibited the highest protein and RNA knockdown (FIGS. 2A-2B). However, the Cas7.1-Cas7.2 truncated construct outperformed all other CTRL-effectors for protein and RNA knockdown at the lower molar ratios (1:1) (FIGS. 2A-2B), only losing 20% of its maximum activity. The protein repression levels by the Cas7.1-Cas7.2 truncated construct did not yield significant variance among different molar ratios (F (3, 12)=1.602, P=0.2407), indicating that the Cas7.1-7.2 variant was more effective at reducing protein expression, which was not affected by the molar ratio. The full-length Cas7-11 protein was sensitive to Effector:Reporter ratios, where a 50% decrease in activity was observed at the lower molar ratios (FIGS. 2A-2B). Interestingly, the Cas7.1 domain exhibited an Effector:Reporter ratio-dependent loss in activity similar to the full-length protein and had significantly more protein expression than Cas7.1-7.2 at all molar ratios (FIGS. 2A-2B).

To confirm whether the gene expression activity was strictly due to DR processing activities, three previously identified residues in the Cas7.1 active site were mutated to alanine (H43A, Y55A, and N152A), to generate Cas7-11 mutants incapable of processing DRs. The results showed that mutations in any of the three active site residues in Cas7.1 restored protein expression (F (3, 8)=3.923, P=0.0542) (FIG. 2C). Mutations in the Cas7.1-Cas7.2 protein, however, still had significantly reduced protein expression compared to the DiCas7-11ΔCas7.1 control (F (4, 10)=387.5, P<0.0001), but protein expression was significantly higher than the unmutated Cas7.1-7.2 construct (F (3, 8)=305.5, P<0.0001) (FIG. 2D). The H43A mutant retained the most RNA knockdown of all mutants (FIG. 2D). Together, these results suggested that C-terminal truncations of Cas7-11 proteins were capable of crRNA processing, the inclusion of Cas11 and Cas7.2 domains improved the efficacy of CTRL at lower molar ratios, and the regulation of protein and RNA expression was strictly driven by Cas7.1-dependent DR processing activity.

### Example 4. CTRL Regulates the Endonuclease Activity of Cas9 and CasRx

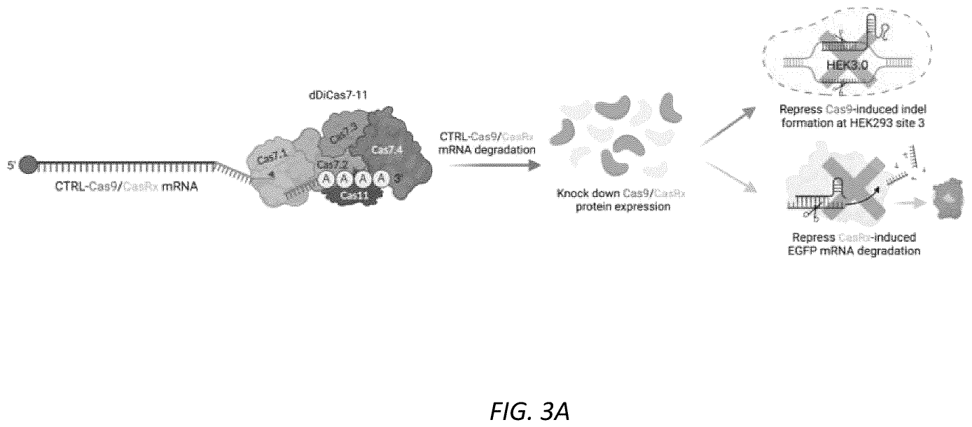

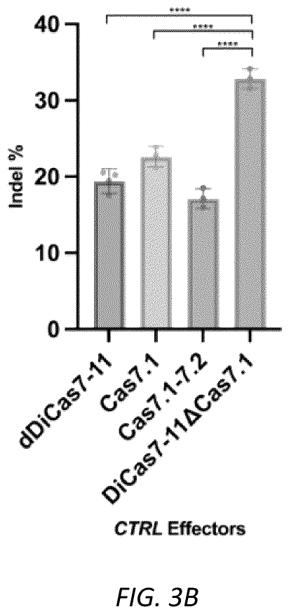

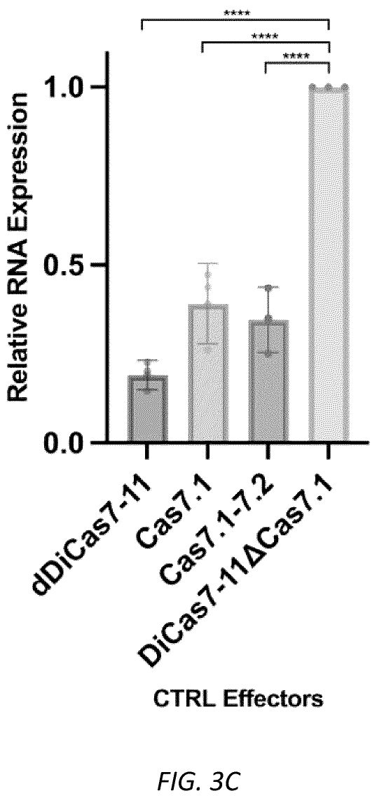

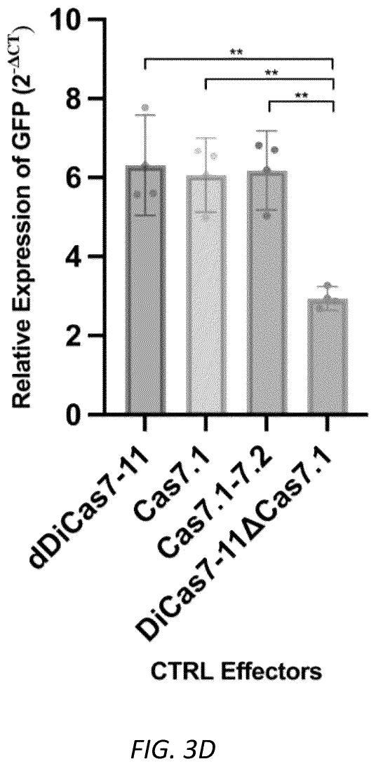

With evidence of RNA and protein regulation by CTRL, the next step was to demonstrate that CTRLs can regulate the activity of a target gene. Toxin-like systems was used to test whether CTRL can reduce the activity of two CRISPR-Cas editors, SpyCas9-NG and RfxCas13d (CasRx). HEK cells were co-transfected with (1) a SpyCas9-NG plasmid including the CTRL-dmDR at the 3′ end and the HEK293-site-3 sgRNA sequence; and (2) a CTRL-effector plasmid (FIG. 3A and FIG. 4). CTRL-effector plasmids and SpyCas9-NG plasmids were simultaneously transfected at a ratio of 15:1 (CTRL-effector:SpyCas9-NG) and amplicon sequencing was performed to analyze the indels generated for each condition. CTRL-effectors reduced SpyCas9-NG indel formation at HEK293-site-3 to 17-21% compared to 31-34% indels in DiCas7-11ΔCas7.1 (FIG. 3B). Of all effectors tested, Cas7.1-7.2 was the most effective and cut SpyCas9-NG indel formation nearly in half (48% reduction). The full-length dDiCas7-11 and Cas7.1 truncated proteins reduced indel formation by approximately 40% and 30%, respectively.

Since indel formation only captures downstream CTRL effects, SpyCas9-NG mRNA expression was also quantified by qPCR. The full-length dDiCas7-11 protein exhibited the largest SpyCas9-NG mRNA knockdown (80%), while Cas7.1 and Cas7.1-7.2 reduced mRNA expression by around 60-65% (FIG. 3C). This result suggested that mRNA knockdown alone did not dictate how effective each CTRL variant would be at reducing the enzymatic activity of SpyCas9-NG.

Next, the ability of CTRL to regulate transient CasRx-mediated gene expression was assessed. Identical to the design of SpyCas9-NG, the CTRL-dmDR was encoded at the 3′ end of the CasRx coding sequence and co-transfected it with CTRL-effector plasmids, EGFP-targeting sgRNA plasmid, and EGFP-expressing plasmid (FIG. 3A and FIG. 4). By competitively targeting CasRx with CTRL, the restoration of EGFP expression was tested. All three CTRL-effectors effectively reduced CasRx expression, increasing EGFP RNA expression 2-fold (FIG. 3D and FIG. 8B). Together, these results demonstrated that CTRL regulated the activity of enzymes that induced both permanent and transient changes in gene expression.

### REFERENCES

1. Makarova, K. S. et al. Evolutionary classification of CRISPR-Cas systems: a burst of class 2 and derived variants. Nature Reviews Microbiology 18, 67-83 (2019).
2. Hale, C. R., Cocozaki, A., Li, H., Terns, R. M. & Terns, M. P. Target RNA capture and cleavage by the Cmr type III-B CRISPR-Cas effector complex. Genes & Development 28, 2432 (2014).
3. Liu, T. Y., Liu, J.-J., Aditham, A. J., Nogales, E. & Doudna, J. A. Target preference of Type III-A CRISPR-Cas complexes at the transcription bubble. Nature Communications 10, 1-13 (2019).
4. O'Connell, M. R. Molecular Mechanisms of RNA Targeting by Cas13-containing Type VI CRISPR-Cas Systems. Journal of molecular biology 431, (2019).
5. Colognori, D., Trinidad, M. & Doudna, J. A. Precise transcript targeting by CRISPR-Csm complexes. Nature Biotechnology 41, 1256-1264 (2023).
6. Ozcan, A. et al. Programmable RNA targeting with the single-protein CRISPR effector Cas7-11. Nature 597, 720-725 (2021).
7. Abudayyeh, O. O. et al. RNA targeting with CRISPR-Cas13. Nature 550, 280-284 (2017).
8. Smargon, A. A. et al. Cas13b is a Type VI-B CRISPR-associated RNA-Guided RNAse differentially regulated by accessory proteins Csx27 and Csx28. Molecular cell 65, 618 (2017).
9. [No title]. https://www.cell.com/cell/pdf/S0092-8674(18)30207-1.pdf.
10. Wessels, H. H. et al. Massively parallel Cas13 screens reveal principles for guide RNA design. Nature biotechnology 38, (2020).
11. Kuo, H.-C., Prupes, J., Chou, C.-W. & Finkelstein, I. J. Massively parallel profiling of RNA-targeting CRISPR-Cas13d. Nature Communications 15, 1-13 (2024).
12. Gootenberg, J. S. et al. Nucleic acid detection with CRISPR-Cas13a/C2c2. Science (New York, N.Y.) 356, 438 (2017).
13. Gootenberg, J. S. et al. Multiplexed and portable nucleic acid detection platform with Cas13, Cas12a, and Csm6. Science (2018) doi: 10.1126/science.aaq0179.
14. Brogan, D. J. et al. Development of a Rapid and Sensitive CasRx-Based Diagnostic Assay for SARS-CoV-2. ACS Sensors (2021) doi: 10.1021/acssensors.1c01088.
15. Buchman, A. B. et al. Programmable RNA Targeting Using CasRx in Flies. The CRISPR Journal 3, 164 (2020).
16. Li, Y. et al. The collateral activity of RfxCas13d can induce lethality in a RfxCas13d knock-in mouse model. Genome Biology 24, 1-25 (2023).
17. Wu, Q.-W. & Kapfhammer, J. P. The Bacterial Enzyme Cas13 Interferes with Neurite Outgrowth from Cultured Cortical Neurons. Toxins 13, 262 (2021).
18. Wang, Q. et al. The CRISPR-Cas13a Gene-Editing System Induces Collateral Cleavage of RNA in Glioma Cells. Advanced Science 6, 1901299 (2019).
19. East-Seletsky, A. et al. Two distinct RNase activities of CRISPR-C2c2 enable guide-RNA processing and RNA detection. Nature 538, 270-273 (2016).
20. Zhang, B. et al. Two HEPN domains dictate CRISPR RNA maturation and target cleavage in Cas13d. Nature Communications 10, 1-11 (2019).
21. Structural Basis for the RNA-Guided Ribonuclease Activity of CRISPR-Cas13d. Cell 175, 212-223.e17 (2018).
22. Kick, L. M., von Wrisberg, M.-K., Runtsch, L. S. & Schneider, S. Structure and mechanism of the RNA dependent RNase Cas13a from Rhodobacter capsulatus. Communications Biology 5, 1-9 (2022).
23. Kato, K. et al. Structure and engineering of the type III-E CRISPR-Cas7-11 effector complex. Cell 185, 2324-2337.e16 (2022).
24. Optimization of specific RNA knockdown in mammalian cells with CRISPR-Cas13. Methods 206, 58-68 (2022).
25. van Beljouw, S. P. B. et al. The gRAMP CRISPR-Cas effector is an RNA endonuclease complexed with a caspase-like peptidase. Science (2021) doi: 10.1126/science.abk2718.
26. Goswami, H. N., Rai, J., Das, A. & Li, H. Molecular mechanism of active Cas7-11 in processing CRISPR RNA and interfering target RNA. Elife 11, (2022).
27. Mugridge, J. S., Coller, J. & Gross, J. D. Structural and molecular mechanisms for the control of eukaryotic 5′-3′ mRNA decay. Nat. Struct. Mol. Biol. 25, 1077-1085 (2018).
28. Lin, C. P. et al. CRISPR RNA binding drives structural ordering that primes Cas7-11 for target cleavage. bioRxivorg (2024) doi: 10.1101/2024.08.01.606276.
29. Brogan, D. J. et al. Synthetic type III-E CRISPR-Cas effectors for programmable RNA-targeting. bioRxiv 2024.02.23.581838 (2024) doi: 10.1101/2024.02.23.581838.
30. Huo, Y., Zhao, H., Dong, Q. & Jiang, T. Cryo-EM structure and protease activity of the type III-E CRISPR-Cas effector. Nat. Microbiol. 8, 522-532 (2023).
31. Nishimasu, H. et al. Engineered CRISPR-Cas9 nuclease with expanded targeting space. Science 361, 1259-1262 (2018).
32. Konermann, S. et al. Transcriptome engineering with RNA-targeting type VI-D CRISPR effectors. Cell 173, 665-676.e14 (2018).
33. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
34. Teixeira, A. P. & Fussenegger, M. Synthetic Gene Circuits for Regulation of Next-Generation Cell-Based Therapeutics. Advanced Science 11, 2309088 (2023).
35. Santos-Moreno, J. & Schaerli, Y. CRISPR-based gene expression control for synthetic gene circuits. Biochemical Society Transactions 48, 1979 (2020).
36. Chen, W. C. W. et al. A synthetic transcription platform for programmable gene expression in mammalian cells. Nature Communications 13, 1-16 (2022).
37. Transcription control engineering and applications in synthetic biology. Synthetic and Systems Biotechnology 2, 176-191 (2017).
38. Houdebine, L.-M., Lema, M. A. & Burachik, M. Advances in the Development of Genetically Modified Animals. Preprint at https://conahcyt.mx/cibiogem/images/cibiogem/comunicacion/publicaciones/CBR-V7/Article2_7.pdf.
39. Yan, Y. et al. Effects of antibiotics on the in vitro expression of tetracycline-off constructs and the performance of Drosophila suzukii female-killing strains. Frontiers in Bioengineering and Biotechnology 11, 876492 (2023).
40. Vasquez, C. A., Cowan, Q. T. & Komor, A. C. Base Editing in Human Cells to Produce Single Nucleotide Variant Clonal Cell Lines. Current protocols in molecular biology 133, e129 (2020).
41. Clement, K. et al. CRISPResso2 provides accurate and rapid genome editing sequence analysis. Nat. Biotechnol. 37, 224-226 (2019).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A recombinant CRISPR-Cas system capable of reducing the expression of one or more target sequence, comprising:
(a) a nucleic acid sequence encoding a CRISPR RNA (crRNA) comprising one or more direct repeats (DRs) and a polynucleotide sequence encoded by the one or more target sequence; and
(b) a Cas peptide comprising a crRNA processing domain,
wherein the recombinant CRISPR-Cas system does not reduce endogenous gene expression.

2. (canceled)

3. The recombinant CRISPR-Cas system of claim 1, wherein the Cas peptide comprising a crRNA processing domain does not have enzymatic cleavage activity.

4. (canceled)

5. The recombinant CRISPR-Cas system of claim 1, wherein the one or more DR is a single non-processed DR, a double non-processed DR (dDR), a double mature DR, or any combination thereof.

6. The recombinant CRISPR-Cas system of claim 1, wherein the single non-processed DR is about 25-45 nucleotides in length.

7. (canceled)

8. The recombinant CRISPR-Cas system of claim 1, wherein the one or more DR comprises a sequence at least 80%, 85%, 90%, 95%, 99%, or 100% identical to SEQ ID Nos.: 33, 34, or 35.

9.-11. (canceled)

12. The recombinant CRISPR-Cas system of claim 1, wherein the target sequence comprises a reporter gene, GFP gene, mCherry gene, Cas 9 gene, CasRx gene, a toxin gene, a synthetic gene, or a transgene.

13. The recombinant CRISPR-Cas system of claim 1, wherein the Cas peptide further comprises a target coordination domain.

14. The recombinant CRISPR-Cas system of claim 1, wherein the target coordination domain is Cas 11, or any other domain of a target coordination activity.

15.-16. (canceled)

17. The recombinant CRISPR-Cas system of claim 1, wherein the Cas peptide comprises a variant of Cas7-11, Cas 12, Cas 13, Cas 5, Cas 6, or fragment thereof, wherein the variant does not have enzymatic cleavage activity.

18. The recombinant CRISPR-Cas system of claim 1, wherein the Cas peptide comprises the crRNA processing domain comprising Cas7.1 domain of a Cas7-11.

19.-26. (canceled)

27. A nucleic acid comprising a polynucleotide encoding recombinant CRISPR-Cas system, or fragments thereof, of claim 1.

28. (canceled)

29. A vector comprising one or more of the nucleic acids of claim 27.

30. A cell comprising the vector of claim 29.

31. (canceled)

32. A pharmaceutical composition comprising the recombinant CRISPR-Cas system of claim 1.

33.-36. (canceled)

37. A method of regulating one or more target sequence expression in a subject, the method comprising:
(a) identifying the one or more target sequence; and
(b) delivering, to the subject, a recombinant CRISPR-Cas system comprising:
a nucleic acid sequence encoding CRISPR-RNA (crRNA) comprising one or more direct repeats and a nucleotide sequence coding the one or more target sequence; and
a type III-E effector comprising a crRNA processing domain,
wherein the type III-E effector and the nucleic acid sequence encoding crRNA are delivered at a ratio of about 1:5 to about 50:1.

38. The method of claim 37, wherein the type III-E effector and the nucleic acid sequence encoding crRNA are delivered at a ratio of about 20:1, 18:1, 16:1, 15:1, 13:1, 11:1, 10:1, 9:1, 8:1, 7:1, 6:1, 5:1, 4:1, 3:1 2:1, 1:1, or 1:2.

39.-44. (canceled)

40. The method of claim 37, wherein delivering the recombinant CRISPR-Cas system comprises delivering a composition comprising a first polynucleotide encoding the crRNA and a second polynucleotide encoding the type III-E effector.

41. (canceled)

42. The method of claim 37, wherein the type III-E effector comprises a crRNA processing domain comprising Cas7.1 of a Cas7-11.

43.-47. (canceled)

48. A method of regulating a toxin gene expression in an insect, the method comprising:
delivering, to the insect, a recombinant CRISPR-Cas system comprising:
a nucleic acid sequence encoding CRISPR-RNA (crRNA) comprising one or more direct repeats and a nucleotide sequence coding the toxin gene, wherein the toxin gene is lethal or nonlethal; and
a type III-E effector comprising a crRNA processing domain,
wherein the type III-E effector and the nucleic acid sequence encoding crRNA are delivered at a ratio of about 1:5 to about 50:1.

49. The method of claim 53, wherein the toxin gene comprises bacterial toxins, diphtheria toxin, botulinum toxin, ricin, cholera toxin, pertussis toxin, Bacillus thuringiensis toxin, or aflatoxins.

50.-64. (canceled)


SEQUENCE LISTING (35 sequences)

SEQ ID NO: 1 - DNA, 23 bp, synthetic construct
  source 1..23: mol_type = other DNA
gtgatcaccgacgagtacaaggt

SEQ ID NO: 2 - DNA, 22 bp, synthetic construct
  source 1..22: mol_type = other DNA
ctggtgtatcttcttctggcgg

SEQ ID NO: 3 - DNA, 18 bp, synthetic construct
  source 1..18: mol_type = other DNA
gctgaagggcatcgactt

SEQ ID NO: 4 - DNA, 19 bp, synthetic construct
  source 1..19: mol_type = other DNA
tgttgtggcggatcttgaa

SEQ ID NO: 5 - DNA, 18 bp, synthetic construct
  source 1..18: mol_type = other DNA
acgtaaacggccacaagt

SEQ ID NO: 6 - DNA, 19 bp, synthetic construct
  source 1..19: mol_type = other DNA
aagaagtcgtgctgcttca

SEQ ID NO: 7 - DNA, 21 bp, synthetic construct
  source 1..21: mol_type = other DNA
ttagtgaaccgtcagatccgc

SEQ ID NO: 8 - DNA, 19 bp, synthetic construct
  source 1..19: mol_type = other DNA
gaacagctcctcgcccttg

SEQ ID NO: 9 - DNA, 19 bp, synthetic construct
  source 1..19: mol_type = other DNA
agggcgaggaggataacat

SEQ ID NO: 10 - DNA, 19 bp, synthetic construct
  source 1..19: mol_type = other DNA
ttggtcaccttcagcttgg

SEQ ID NO: 11 - protein, 1601 aa, synthetic construct
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTALQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTAVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 12 - protein, 237 aa, Desulfonema ishimotonii
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRF

SEQ ID NO: 13 - protein, 581 aa, synthetic construct
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTALQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLD

SEQ ID NO: 14 - protein, 1364 aa, synthetic construct
DEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 15 - skipped

SEQ ID NO: 16 - protein, 237 aa, synthetic construct
MTTTMKISIEFLEPFRMTKWQESTRRNKNNKEFVRGQAFARWARNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRF

SEQ ID NO: 17 - protein, 237 aa, synthetic construct
MTTTMKISIEFLEPFRMTKWQESTRRNKNNKEFVRGQAFARWHRNKKDNTKGRPAITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRF

SEQ ID NO: 18 - protein, 237 aa, synthetic construct
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGALSLPGKPDFDGPKAIGSQRVLNRVDFKSGKAHDFFKAYEVDHTRFPRFEGEITIDNKVSAEARKLLCDSLKFTDRLCGALCVIRF

SEQ ID NO: 19 - protein, 581 aa, synthetic construct
MTTTMKISIEFLEPFRMTKWQESTRRNKNNKEFVRGQAFARWARNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTALQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLD

SEQ ID NO: 20 - protein, 581 aa, synthetic construct
MTTTMKISIEFLEPFRMTKWQESTRRNKNNKEFVRGQAFARWHRNKKDNTKGRPAITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTALQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLD

SEQ ID NO: 21 - protein, 581 aa, synthetic construct
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGALSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTALQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLD

SEQ ID NO: 22 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 23 - RNA, 13 nt, synthetic construct
  source 1..13: mol_type = other RNA
gccgccrccatgg

SEQ ID NO: 24 - DNA, 249 bp, synthetic construct
  source 1..249: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 25 - DNA, 701 bp, synthetic construct
  source 1..701: mol_type = other DNA
atacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaaccctt

SEQ ID NO: 26 - DNA, 103 bp, synthetic construct
  source 1..103: mol_type = other DNA
ggcccagactgagcacgtgagtttaagagctagaaatagcaagtttaaataaggctagtccgttatcaacttgaaaaagtggcaccgagtcggtgcttttttt

SEQ ID NO: 27 - protein, 1601 aa, Desulfonema ishimotonii
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 28 - protein, 181 aa, Desulfonema ishimotonii
KETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDL

SEQ ID NO: 29 - protein, 200 aa, Desulfonema ishimotonii
WHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKG

SEQ ID NO: 30 - protein, 89 aa, Desulfonema ishimotonii
LTGKIRCKLITKTPLIVPDTSNDDFFRPADKEARKEKDEYHKSYAFFRLHKQIMIPGSELRGMVSSVYETVTNSCFRIFDETKRLSWRM

SEQ ID NO: 31 - protein, 214 aa, Desulfonema ishimotonii
RTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRK

SEQ ID NO: 32 - protein, 108 aa, Desulfonema ishimotonii
NLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMS

SEQ ID NO: 33 - DNA, 35 bp, synthetic construct
  source 1..35: mol_type = other DNA
ggttggaaagccggttttctttgatgtcacggaac

SEQ ID NO: 34 - DNA, 92 bp, synthetic construct
  source 1..92: mol_type = other DNA
ggttggaaagccggttttctttgatgtcacggaacctacctggtagcccttgtatttggttggaaagccggttttctttgatgtcacggaac

SEQ ID NO: 35 - DNA, 52 bp, synthetic construct
  source 1..52: mol_type = other DNA
ttgatgtcacggaacctacctggtagcccttgtatttttgatgtcacggaac